(19) United States
(12) Patent Application Publication
(10) Pub. No.: US 2006/0014151 A1
(43) Pub. Date: Jan. 19, 2006

(54) Optical dna sensor, dna reading apparatus, identification method of dna and manufacturing method of optical dna sensor

(76) Inventors: Jun Ogura, Tokyo (JP); Hideaki Ishida, Tokyo (JP)

Correspondence Address: FRISHAUF, HOLTZ, GOODMAN & CHICK, PC, 220 5TH AVE FL 16, NEW YORK, NY, 10001-7708, US

(21) Appl. No.: 10/534,368

(22) PCT Filed: Dec. 18, 2003
(86) PCT No.: PCT/JP03/16227

§ 371 Date: May 9, 2005

(30) Foreign Application Priority Data
Dec. 25, 2002 (JP) 2002-374695
Dec. 25, 2002 (JP) 2002-374794

(51) Int. Cl. C12Q 1/68; C12M 1/34

(52) U.S. Cl. 435/6; 435/287.2

(57) ABSTRACT

The advantage is to provide a DNA reading apparatus which can sense fluorescence even if the sensitivity of a CCD image sensor or a photomul is low and can be constructed in a compact size. An optical DNA sensor having: a solid imaging device, and a plurality types of DNA probe each including nucleotide sequence and being arrayed and fixed on a surface of the solid imaging device.

### FIELD OF THE INVENTION

The present invention relates to an optical DNA sensor used for determining a DNA sequence and a method for manufacturing the same, and to a DNA reading apparatus using the DNA sensor and a method for identification of DNA.

### BACKGROUND OF THE INVENTION

In recent years, gene information about living organism has been utilized in wide range of fields such as medical field and agricultural field. However, it is indispensable to elucidate DNA sequences in order to utilize genes. DNA includes two polynucleotide chains that are helically twisted, each of the polynucleotide chains comprises a polynucleotide sequence in which four bases (adenine: A, guanine: G, cytosine: C, and Thymine: T) are linearly arrayed. Those bases in one polynucleotide chain respectively bind to bases in the other polynucleotide chain in accordance with complementarities between adenine and thymine and between guanine and cytosine.

The expression of the elucidation of DNA sequence denotes an operation to specify a sequence of nucleotides. For determining the nucleotides sequence, a DNA micro array and a reading apparatus thereof have been developed. A nucleotide sequence of a sample DNA is specified as follows by means of a DNA micro array and the reading apparatus thereof.

First, DNA micro array prepared by aligning and fixing each of a plurality types of DNA probes in which a known nucleotide sequence is included, on a solid carrier such as a glass slide, is prepared. Then, a sample DNA including an unknown nucleotide sequence is denatured to a single strand DNA segment, and the denatured sample DNA segment is bonded with a fluorescent substance or the like.

Then, the sample DNA segment is added onto the DNA micro array. As a result, the sample DNA segment hybridizes to a DNA probe including a mutually-complementary nucleotide sequence. Specifically, each of bases included in the sample DNA segment binds by hydrogen bond to each of bases included in the complementary DNA segment selected among the plurality types of DNA probe to then form a double strand of the sample DNA and the DNA probe. On the other hand, the sample DNA segment does not bind to a DNA probe that is not complementary therewith. Under a condition that the sample DNA segment has been marked with a fluorescent substance, fluorescence is emitted in the vicinity of the DNA probe having bonded with the sample DNA segment, when light that excites the fluorescent substance is irradiated to the sample DNA segment. For example, in case of a sample DNA segment including a nucleotide sequence TCGGGAA bonds only with a DNA probe that includes a nucleotide sequence AGCCCTT, and the fluorescent substance applied to the sample DNA segment having bonded with that DNA probe emits fluorescence.

Then, the DNA micro array is set to a reading apparatus, and the DNA segment therein is analyzed by the reading apparatus. The reading apparatus is a type to measure fluorescence intensity distribution on the DNA micro array.

There are two major types of the reading apparatuses, that is, of an evanescent system and of a confocal laser system, for example, disclosed in Japanese Patent Publication (Laid-open) No. Hei 9-23900.

The reading apparatus of the evanescent system is constituted such that, when excited light is irradiated from a lateral side of a DNA micro array substrate, evanescent light having exuded slightly on the surface of the substrate excites a fluorescent substance applied to complementarily bonded DNA to cause the fluorescent substance to emit light, and the emitted light is received by a photodiode, thereby allowing the photodiode to determine the position of the complementary DNA probe.

The DNA reading apparatus of the confocal laser system is constituted such that laser beams obtained by converging light emitted from a laser diode by means of a collimator lens is irradiated to one point on a DNA micro array, this point light is scanned in a cross direction of the array, a photomultiplier tube (photomul) is scanned simultaneously with two-dimensional scanning of the laser beam, fluorescence emitted by the irradiation of the laser beam is received by the photomul to measure fluorescence intensity, thereby determining the fluorescence intensity distribution in the surface of the DNA micro array.

With the DNA reading apparatus of either system described above, the fluorescence intensity distribution on the DNA micro array is outputted as an image of two dimensions. In the fluorescence intensity distribution, it is shown that the DNA probe including a nucleotide sequence of the sample DNA segment and the complementary nucleotide sequence thereof is contained in a part where the fluorescence intensity is greater in the outputted image. Hence, it is possible to determine the nucleotide sequence of the sample DNA segment by checking the parts with greater fluorescence intensity in the two-dimensional image.

However, the DNA reading apparatus of the confocal laser system has a large mechanism in size, that controls the focus of an optical lens interposed between the laser beam and the DNA micro array and adapted to make the laser beam into a point beam and to scan on the micro array, and scans even areas in between the adjacent DNA probe, where no DNA probe exists. As a result, the DNA reading apparatus of this system has a drawback that it requires a longer period of time for the scanning. The DNA reading apparatus of the evanescent system requires a light source in the lateral direction since it irradiates light from the lateral side of the DNA micro array. As a result, it has such a drawback that the width thereof becomes longer and the size thereof becomes larger.

With a conventional DNA reading apparatus of either of the foresaid systems, the fluorescence intensity is also sensed in an area between adjacent DNA probe on a DNA micro array. As a result, data of the fluorescence intensity for unnecessary parts on the DNA micro array, where no DNA probe is arranged, is also included in the images.

Furthermore, fluorescence intensity emitted from the DNA probe having been bonded to the sample DNA segment is not always high, and a CCD image sensor and a photomul are remote from the DNA micro array. As a result, it is required to increase the sensitivities of the CCD image sensor and photomul.

### SUMMARY OF THE INVENTION

Therefore, it is an advantage of the DNA reading apparatus according to the present invention that it can sense fluorescence even if the sensitivity of an optical DNA sensor is low and can be constructed in a compact size.

An optical DNA sensor according to the present invention comprises:
a solid imaging device, and
a plurality of types of DNA probe each including nucleotide sequence and being arrayed and fixed on the surface of the solid imaging device.

According to the present invention, clear images can be imaged by means of the solid imaging device without being provided with lenses or microscopes, and further, images of two dimensions can be imaged without being provided with a scanning mechanism, by means of the solid imaging device. Therefore, when the optical DNA sensor according to the present invention is used in a DNA reading apparatus, it becomes needless to provide the reading apparatus with a lens, a microscope and a scanning mechanism. As a result, the DNA reading apparatus can be constructed in a compact size smaller in comparison with the size of the conventional similar apparatuses. In addition, according to the present invention, light emitted from the DNA probe can be incident to the surface of the solid imaging device substantially without causing attenuation. Therefore, the sensitivity of the solid imaging device needs not to be so high.

Alternatively, the optical DNA sensor according to the present invention comprises:
a solid imaging device,
an excited light absorbing layer formed on the surface of the solid imaging device, and
A plurality types of DNA probe-which include nucleotide sequence and are aligned and fixed on the excited light absorbing layer.

According to this invention, difference in brightness between the part of the DNA probe bonded to the sample DNA segment and the part of the DNA probe not bonded to the sample DNA segment becomes clear, whereby images with high contrast can be imaged by means of the solid imaging device. Accordingly, it becomes possible to easily determine which part in the images imaged by the solid imaging device is greater in the intensity, and determination of the nucleotide sequence in the sample DNA segment can be facilitated.

Further, when the DNA probe are fixed on the transparent layer such that each one of them corresponds to one of the photoelectric conversion elements, respectively, intensity of light on the areas between every two of the DNA probe never be sensed. Therefore, images imaged by the solid imaging device have no noise and does not contain data on the intensity of light for the parts where the DNA probe are not arranged.

By constituting the photoelectric conversion elements into field-effect transistor type elements each including a semiconductor layer that generates charges when it is irradiated with light, switching and the like of electric signals in pixels can be performed with only the photoelectric conversion elements. As a result, not only the photoelectric conversion elements but also the DNA probe can be arrayed in a high density, respectively.

According to the DNA reading apparatus of the present invention, it is needless to provide the DNA reading apparatus with lenses and microscopes for image-forming the part in which the DNA probe are arrayed on the solid imaging device. As a result, manufacturing of the DNA reading apparatus in a compact size is enabled.

According to the DNA identification method of the present invention, light emitted from the DNA probe is incident to the photoelectric conversion elements substantially without causing attenuation. As a result, it is possible to recognize the difference between the intensity of the light emitted from the complementary DNA segment and the intensity of the light emitted from the DNA segment being not complementary even if the sensitivity of the photoelectric element is not so high. This makes the identification of the sample DNA segment easy.

According to the manufacturing method of the solid imaging device of the present invention, the DNA probe are drawn to the surface of the solid imaging device by the force of static electricity, whereby it becomes easy to fix the DNA probe on the surface of the solid imaging device.

### BRIEF DESCRIPTION OF THE DRAWINGS

FIG. 1 is a perspective view showing an optical DNA sensor to which the present invention is applied;

FIG. 2 is a plan view showing the optical DNA sensor of FIG. 1;

FIG. 3 is a cross-section of the optical DNA sensor when it is cut along a broken line (III)-(III) indicated in FIG. 2;

FIG. 4A is a plan view showing a pixel of a solid imaging device included in the optical DNA sensor of FIG. 1, and FIG. 4B is a cross-section of the pixel when it is cut along a broken line (IVB)-(IVB) indicated in FIG. 4A;

FIG. 5 is a view showing a circuit configuration of a DNA reading apparatus using the optical DNA sensor of FIG. 1;

FIG. 6 is a view showing a structure of the DNA reading apparatus on which the optical DNA sensor of FIG. 1 is set;

FIG. 7 is an oblique perspective view showing a mother substrate comprising a plurality of solid imaging devices;

FIG. 8 is a timing chart showing a succession of levels of electric signals outputted by drivers of the solid imaging devices;

FIG. 9 is a plan view showing an optical DNA sensor different from the aforementioned optical DNA sensor;

FIG. 10 is a cross-section of the optical DNA sensor of FIG. 9 when it is cutting along a broken line (X)-(X) indicated in FIG. 9;

FIG. 11 is a cross-section of an optical DNA sensor according to the third embodiment;

FIG. 12A is a plan view showing one of pixels of a solid imaging device included in an optical DNA sensor according to the third embodiment, and FIG. 12B is a cross-section of the pixel of FIG. 12A when it is cut along a broken line (XIIB)-(XIIB) indicated in FIG. 12A;

FIG. 13A is a view showing wavelength dependence of photosensitivity of amorphous silicon, FIG. 13B is a logarithmic graph showing a relation between a thickness of an excited light absorbing layer 34 formed on the surface of the solid imaging device and a transmittance of phosphor exciting light and fluorescence, and FIG. 13C is a logarithmic graph showing a relation between a thickness of the excited light absorbing layer 34 when a charge density of ITO is further controlled and a transmittance of phosphor exciting light and fluorescence;

FIG. 14 is a view showing a structure of the DNA reading apparatus on which the optical DNA sensor according to the third embodiment is set;

FIG. 15 is a plan view showing a relation between a charge density of tin-doped indium oxide and a light absorption wavelength end;

FIG. 16A is a plan view showing a pixel of a solid imaging device included in an optical DNA sensor different from the optical DNA sensor of FIG. 1, and FIG. 16B is a cross-section of the pixel of FIG. 16A when it is cut along a broken line (XVIB)-(XVIB) indicated in FIG. 16A;

FIG. 17 is a view showing a structure of a DNA reading apparatus different from the DNA reading apparatus of FIG. 14, on which an optical DNA sensor is set; and

FIG. 18 a view showing a structure of a DNA reading apparatus different from the DNA reading apparatuses of FIGS. 14 and 17, on which an optical DNA sensor is set.

### PREFERRED EMBODIMENT OF THE INVENTION

Some specific embodiments of the present invention are explained below with reference to the appended drawings. However, it should be noted that it is not intended to limit the scope of the present invention to the examples shown in the drawings.

### First Embodiment

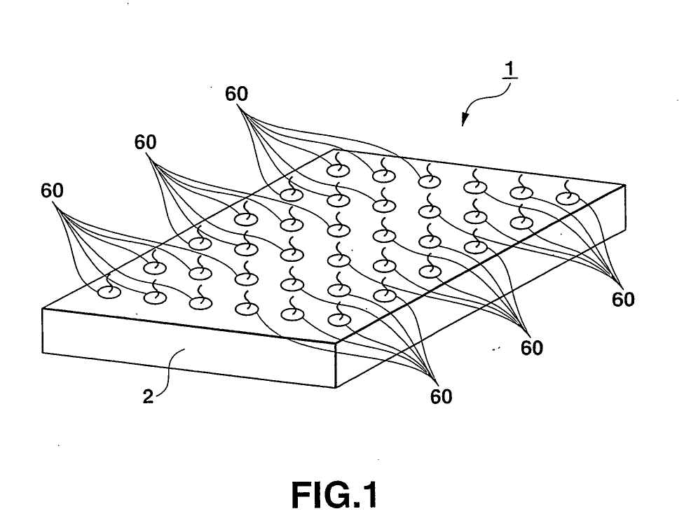

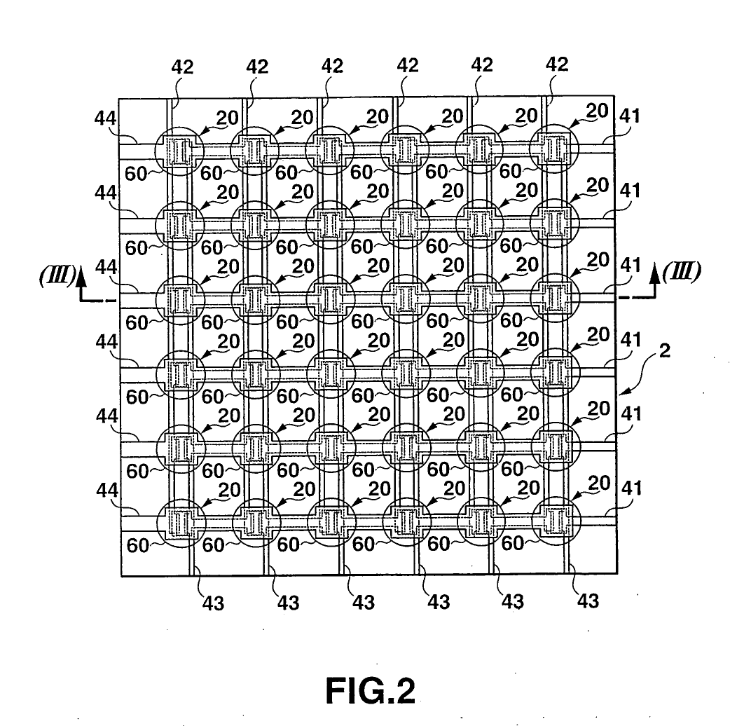

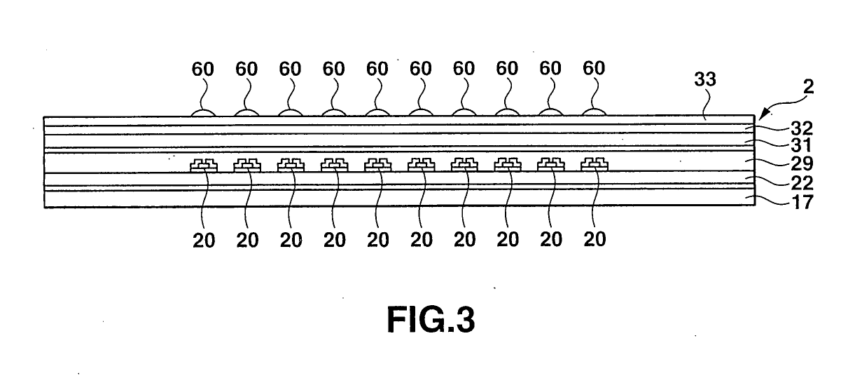

FIG. 1 is an oblique perspective view showing an optical DNA sensor to which the present invention is applied, FIG. 2 is a plan view of the optical DNA sensor, and FIG. 3 is a cross-section of the sensor when it is cut along a broken line (III)-(III) in FIG. 2 and observed to a direction indicated by arrows.

An optical DNA sensor 1 includes a solid imaging device 2 and spots 60, 60, . . . collocated and fixed on a surface of the solid imaging device 2, and in which each of the spots 60 is configured to correspond to each of pixels of the solid imaging device 2.

First, the solid imaging device will be explained below. The solid imaging device 2 includes a transparent substrate 17 of a substantially flat plate shape, photo-sensor elements (hereinafter referred to as sensors) 20, 20, . . . including a plurality of double gate type field-effect transistors those which are arranged in a matrix fashion consisting of n lines and m rows, (wherein both n and m are a positive integer) on one of surfaces of the transparent substrate 17, a protective insulated layer 31 for coating all the sensors 20, 20, . . . in the block, and a conductive layer 32 formed on the protective insulated layer 31. Both the protective insulated layer 31 and conductive layer 32 are transparent.

The transparent substrate 17 is light-permeable to light of a wavelength range of 350 to 1,000 nm, which covers a range of from ultraviolet rays to visible rays, (hereinafter referred to simply as light permeability), and has insulating property, and it is a glass substrate such as silica glass or a plastic substrate such as polycarbonate. This transparent substrate 17 comprises the reverse face of the solid imaging device 2. Note that a substrate having shading property may be used instead of the transparent substrate 17 having light-permeable property.

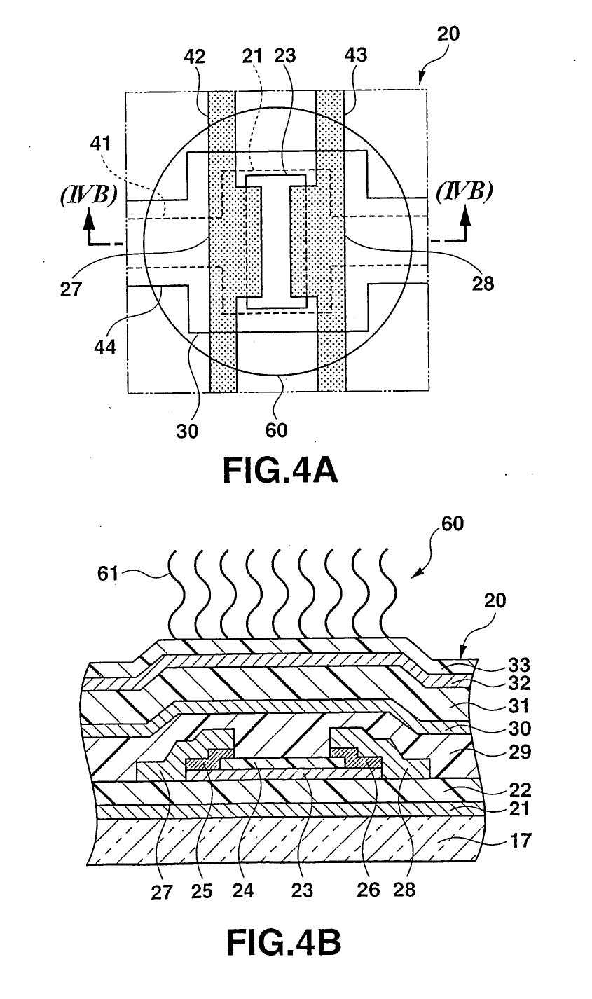

Now, the sensor 20 will be described below. FIG. 4A is a plan view showing one of the sensors 20, and FIG. 4B is a cross-section of the sensor when it is cut along a broken line (IVB)-(IVB) and observed to a direction indicated by arrows.

Each of the sensors 20 is a photoelectric element functioning as a pixel. Each of the sensors 20 comprises a bottom gate electrode 21 formed on the transparent substrate 17, a bottom gate insulated film 22 formed on the bottom gate electrode 21, a semiconductor layer 23 that clamps the bottom gate insulated film 22 in between the bottom gate electrode 21 and faces to the bottom gate electrode 21, a channel protective film 24 formed on the central portion of the semiconductor layer 23, impurities semiconductor layers 25, 26 formed on both end portions of the semiconductor layer 23 and being apart from each other, a source electrode 27 formed on the impurities semiconductor layer 25, a drain electrode 28 formed on the impurities semiconductor layer 26, a top gate insulated film 29 formed on the source electrode 27 and drain electrode 28, and a top gate electrode 30 that clams the top gate insulated film 29 and channel protective film 24 in between the semiconductor layer 23 and faces to the semiconductor layer 23.

In each of the sensors 20, a bottom gate electrode 21 is formed on the transparent substrate 17. Besides, on the transparent substrate 17, n pieces of bottom gate lines 41, 41, . . . are formed so as to extend in the lateral direction, and the bottom gate electrode 21 of each of the sensors 20 on the same line arrayed in the lateral direction is formed with a common bottom gate line 21 in one united body. Both of the bottom gate electrode 21 and bottom gate line 41 have conductive and shading properties and they are made from, for example, chromium, a chromium alloy, aluminum or an aluminum alloy, or an alloy thereof, respectively.

On the bottom gate electrode 21 and bottom gate line 41, a bottom gate insulated film 22 that is common to all of the sensor 20, 20, . . . is formed. The bottom gate insulated film 22 has insulating and light-transmitting properties and is made from, for example, silicon nitride (SiN) or silicon oxide (SiO₂).

In each of the sensor 20, a semiconductor layer 23 is formed on the bottom gate insulated film 22. The semiconductor layer 23 is substantially rectangular-shaped in the plan view and is a film made of amorphous silicon or polysilicon that is not excited sufficiently when receiving ultraviolet rays (of a wavelength range of less than 400 nm) but is excited sufficiently when receiving visible light of a longer wavelength (400 nm or longer) to generate electron-hole pairs corresponding to the amount of the light. The channel protective layer 24 is formed on the semiconductor layer 23. The channel protective film 24 has a function to protect the interface of the semiconductor-layer 23 from an etchant-used for patterning and insulating and light-transmitting properties, and it comprises, for example, silicon nitride or silicon oxide. When light is incident to the semiconductor layer 23, the electron-hole pairs in an amount in accordance with the amount of the incident light are generated-inside the semiconductor layer 23.

On the one end portion of the semiconductor layer 23, the impurities semiconductor layer 25 is formed such that the part thereof is superimposed on the channel protective film 24. On the other end portion of the semiconductor layer 23, the impurities semiconductor layer 26 is formed such that the part thereof is superimposed on the channel protective film 24. The impurities semiconductor layers 25, 26 are patterned for each sensor 20. The impurities semiconductor layers 25, 26 respectively comprise amorphous silicon containing n-type impurity ions (n+ silicon).

A source electrode 27 patterned for each sensor 20 is formed on the impurities semiconductor layer 25. Besides, a drain electrode 28 patterned for each sensor 20 is formed on the impurities semiconductor layer 26. Furthermore, m pieces of source lines 42, 42, . . . and data lines 43, 43, . . . , those which are extending in the longitudinal direction, are formed on the bottom gate insulated film 22. The source electrode 27 in each of the sensors 20 on the same row arrayed in the longitudinal direction is formed with a common source line 42 in one united body, and the drain electrode 28 in each of the sensors 20 on the same row arrayed in the longitudinal direction is formed with a common data line 43 in one united body. The source electrode 27, the drain electrode 28, the source line 42 and the data line 43 respectively have conductive and shading properties, and they respectively comprise, for example, chromium, a chromium alloy, aluminum or an aluminum alloy, or an alloy thereof.

On the channel protective films 24, the source electrodes 27 and the drain electrodes 28 of all of the sensors 20, 20, . . . and the source lines 42, 42, . . . and the data lines 43, 43, . . . , the top gate insulated film 29 that is common to all of the sensors 20, 20, . . . is formed. The top gate insulated film 29 has insulating and light-transmitting properties and comprises, for example, silicon nitride or silicon oxide.

On the top gate insulated film 29, a top gate electrodes 30 patterned for each sensor 20 is formed. In addition, n pieces of top gate lines 44 that extend in the lateral direction are formed on the top gate insulated film 29. The top gate electrode 30 in each of the sensors 20 on the same row arrayed in the lateral direction is formed with a common top gate line 44 in one united body. The top gate electrode 30 and the top gate lines 44 respectively have conductive and light-transmitting properties and they respectively comprise, for example, indium oxide, zinc oxide or tin oxide, or a mixture containing at least one thereof (e.g., tin-doped indium oxide (ITO), zinc-doped indium oxide).

The sensor 20 constructed as described above is a photoelectric conversion element including the semiconductor layer 23 functioning as a light reception section.

On the top gate electrodes 30 and the top gate lines 44, 44, . . . of all of the sensors 20, 20, . . . , a common protective insulated layer 31 is formed such that it coats the top gate electrodes 30 and the top gate lines 44. The protective insulated layer 31 has insulating and light-transmitting properties and comprises silicon nitride or silicon oxide.

Throughout on the protective insulated layer 31, a conductive layer 32 is formed. The conductive layer 32 has conductive and light-transmitting properties and comprises, for example, indium oxide, zinc oxide or tin oxide, or a mixture comprising at least one thereof.

Throughout on the conductive layer 32, an overcoat layer is formed. This overcoat layer 33 has light-transmitting property and functions so as to protect the conductive layer 32 and fix the spots 60, 60, . . . onto the surface of the solid imaging device 2.

Next, description on the spot 60 will be given below. As shown in FIGS. 1 to 3, a plurality types of spots 60, 60, . . . are arrayed in a matrix fashion consisting of n lines and m rows on the overcoat layer 33 such that they are remote from one to another. One of the spots 60 is a concourse of a large number of DNA probe 61 of a single strand. A large number of DNA probe 61 contained in a spot 60 have the same nucleotides sequence, respectively. The configurations of the nucleotides sequences in the DNA probe of a single strand differ for every spot 60 from one to another. In the nucleotide sequences of any spots 60, the nucleotide sequence have been known.

The spots 60, 60, . . . as described above are arrayed respectively corresponding to each of the sensors 20, 20, . . . Specifically, as shown in FIGS. 2 and 4A, when the solid imaging device is viewed in the plan view, it is configured such that one spot 60 is superimposed on one sensor 20. In particular, the semiconductor layer 23 of the sensor 20 is superimposed on one spot 60.

As a method to fix the spots 60, 60, . . . onto the surface of the solid imaging device 2, a method comprising steps of: attaching DNA probe 61 prepared in advance in droplets onto the surface of the solid imaging device 2 by means of a dispenser, applying the surface of the solid imaging device with polycation (such as poly-L-lysine, poly(ethylene imine) and the like), and utilizing charges of DNA to bond the spots to the surface of the solid imaging device 2 thanks to the electrostatic bond is applied.

As the other fixing method, a way to use a silane coupler including amino, aldehyde, epoxy and the like has been also employed. In case of using such a method, the amino, aldehyde and the like are introduced to the surface of the solid imaging device thanks to their covalent bond. Therefore, the spots can stably exist on the surface of the solid imaging device comparing to the fixing by using the polycation.

In addition, there is also another method for the fixing, wherein an oligonucleotide to which a labile group is introduced is synthesized, the oligonucleotide in a droplet is attached to the surface of the surface-treated solid imaging device 2, and the oligonucleotide is then bonded to the surface thanks to their covalent bond.

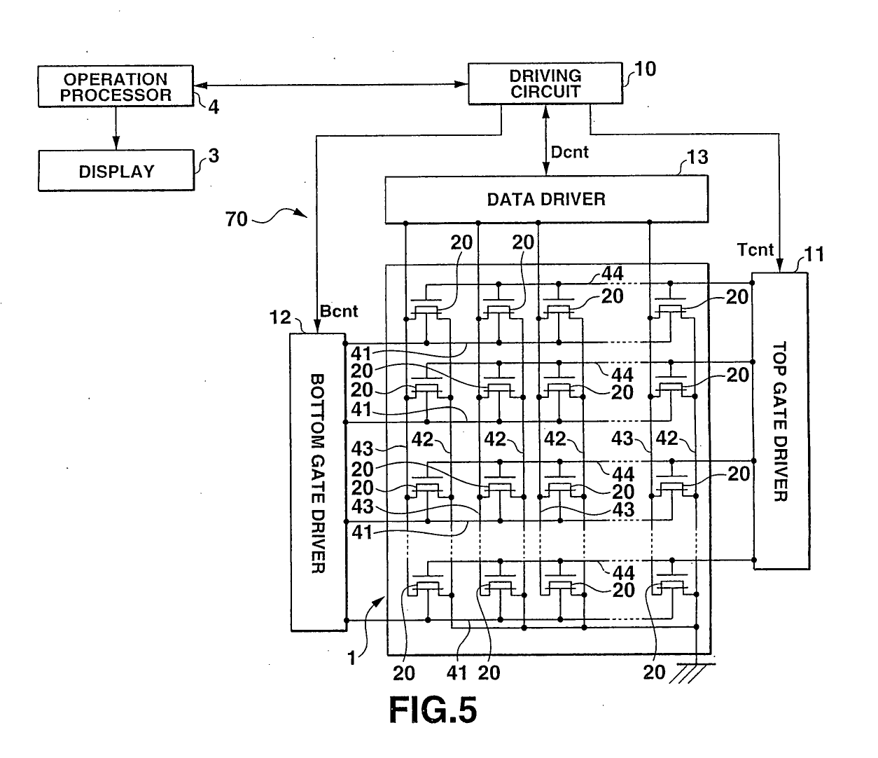

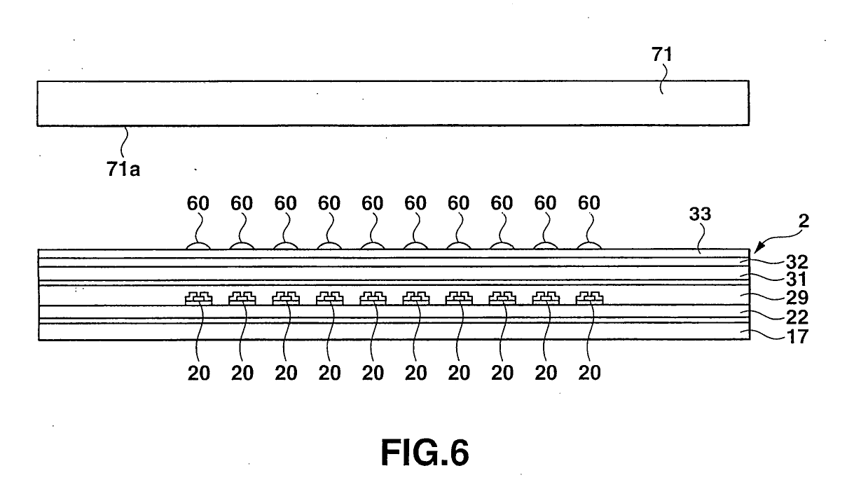

Next, the DNA reading apparatus in which the optical DNA sensor constituted as described above will be described with reference to FIGS. 5 and 6.

As shown in FIGS. 5 and 6, the DNA reading apparatus 70 comprises a display 3, an operation processor 4 for controlling the whole apparatus, a light irradiation means 71 for irradiating phosphor exciting light in the form like a plane of light from the vicinity to the surface of the optical DNA sensor 1, and a driving means (consisting of a top gate driver 11, a bottom gate driver 12, a data driver 13 and a driving circuit 10) for driving the optical DNA sensor 1 to acquire images.

The light irradiation means 71 includes a light source that does not include a wavelength range of sufficiently exciting the semiconductor layer 23 but emits phosphor exciting light (mainly ultraviolet rays) in a wavelength range of sufficiently exciting a fluorescent substance described later and a prism or a band-shaped optical fiber bundle that totally reflects the phosphor exciting light emitted from the light source to thereby emit the phosphor exciting light in the vicinity from the total reflection surface to the exterior. The optical DNA sensor 1 is constructed such that it can be attached to and removed from the DNA reading apparatus 70, and the surface of the solid imaging device 2 of the optical DNA sensor 1 attached to the DNA reading apparatus 70 is arranged so as to be adjacent and face to the projection surface 71a (total reflection surface) of the phosphor exciting light. When the optical DNA sensor 1 had faced to the projection surface 71a of the light irradiation means 71, it is constituted that the phosphor exciting light in the vicinity that is irradiated from the projection surface is uniformly irradiated to the surface of the solid imaging device 2. The solid imaging device 2 of the optical DNA sensor 1 neither show its sensitivity to the phosphor exciting light that is irradiated out from the projection surface 71a nor be excited. However, the solid imaging device shows its sensitivity to fluorescence (mainly visible light) emitted from a fluorescent substance when it is irradiated with the phosphor exciting light and is excited at the same time.

The DNA reading apparatus is configured such that, when the optical DNA sensor 1 is attached to the DNA reading apparatus 70, the top gate lines 44, 44, . . . of the optical DNA sensor 1 are connected to the terminals of the top gate driver 11, respectively. Similarly, the bottom gate lines 41, 41, . . . of the optical DNA sensor 1 are connected to the terminals of the bottom gate driver 12, respectively, and the data lines 43, 43, . . . of the optical DBA sensor 1 are connected to the terminals of the data driver 13, respectively. Besides, when the optical DNA sensor 1 is attached to the DNA reading apparatus 70, the source lines 42, 42, . . . of the optical DNA sensor 1 are connected to a specific voltage source. In this embodiment, the source lines are connected to the earth.

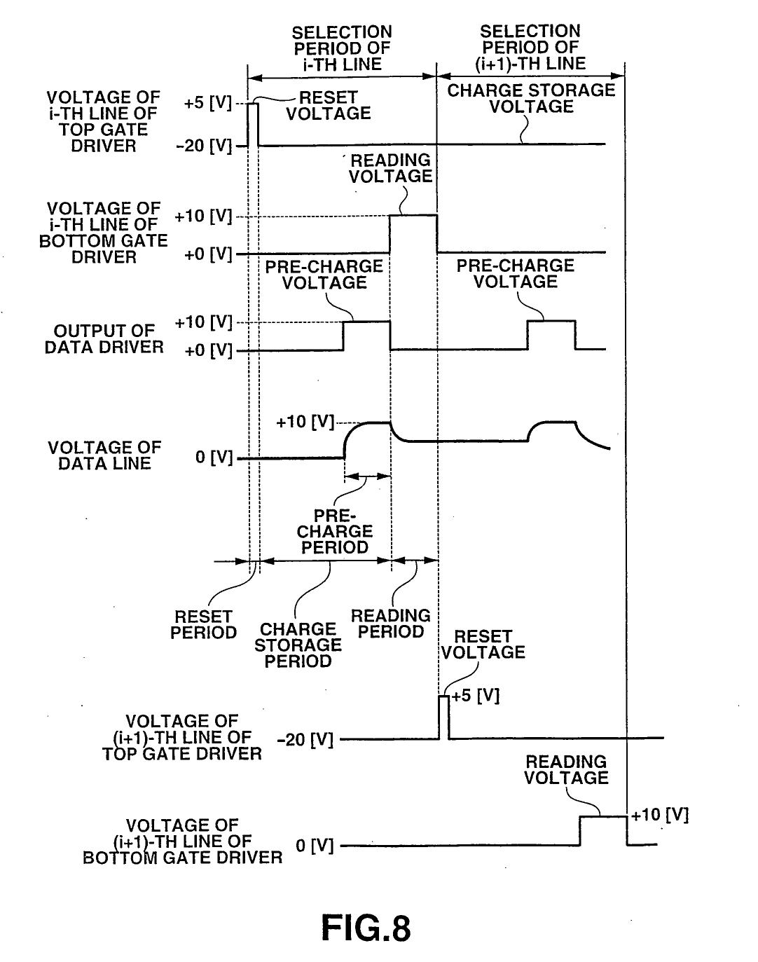

The top gate driver 11 is a shift register. That is to say, the top gate driver 11 is configured to input a control signal Tcnt from the driving circuit 10 to thereby output a reset voltage (shown in FIG. 8) in order of from the top gate line 44 of the first line to the top gate line 44 of the n-th line (when reached to the n-th line, return to the first line upon requirement). The level of the reset voltage is at a high level of +5 [V]. On the other hand, the top gate driver 11 is configured to apply an electric potential at a low level of −20 [V] to the respective top gate lines 44 when it does not output the reset voltage.

The bottom gate driver 12 is a shift register. Specifically, the bottom gate driver 12 is configured to output a control signal Bcnt from the driving circuit 10 to thereby output a read voltage (shown in FIG. 8) in order of from the bottom gate line 41 of the first line to the bottom gate line of the n-th line (when reached to the n-th line, return to the first line upon requirement). The level of the read voltage is at a high level of +10 [V], and the level of the read voltage when it is not outputted is at a low level of ±0 [V].

The top gate driver 11 and the bottom gate driver 12 are configured to shift output signals such that, after the top gate driver 11 outputted the reset voltage to the top gate line 44 of the i-th line (i is an integer of 1 to n) and a certain charge storage period has then elapsed, the bottom gate driver 12 outputs the read voltage to the bottom gate line of the i-th line. That is, in each of the lines, the timing for the read voltage to be outputted is delayed from the timing for the reset voltage to be outputted. Besides, the period from the start of an input of the reset voltage to the top gate line 44 of the i-th line (i is any of 1 to n) to the end of an input of the read voltage to the bottom gate line 41 of the i-th line is a selection period of time of the i-th line. The level of the reset voltage is at a high level of +5 [V], and the level of the reset voltage when it is not outputted is at a low level of −20 [V].

The data driver 13 is configured to output a pre-charge voltage (shown in FIG. 8) to all of the data lines 43, 43, . . . during a period from completion of the output of the reset voltage until completion of the output of the read voltage in the selection period of time of each line. The level of the pre-charge voltage is at a high level of +10 [V], and the level of the pre-charge voltage when it is not outputted is at a low level of +0 [V]. Besides, the data driver 13 is configured to amplify the voltage of the data lines 43, 43, . . . following to the output of the pre-charge voltage to output the voltage to the driving circuit 10.

The driving circuit 10 is configured to be driven by the operation processor 4 to output control signals Bcnt, Tcnt and Dcnt to each of the bottom gate driver 12, top gate driver 11 and data driver 13, whereby causing them properly to output voltages upon requirements. Further, the driving circuit 10 is configured to detect voltages of the data lines 43, 43, . . . after a preset time elapse following to the output of the read voltage or to detect the period of time from outputting the read voltage until the time at which the voltages of the data lines 43, 43, . . . reach to a preset threshold voltage to thereby acquire images and output the images to the operation processor 4. The operation processor 4 is configured to display the images inputted from the driving circuit 10 on a display 3.

Since the spots 60, 60, . . . are arrayed on the surface of the solid imaging device 2 as described above, clear images can be imaged by the solid imaging device 2 without providing the DNA reading apparatus 70 with an optical system such as lenses and microscopes. Accordingly, the DNA reading apparatus can be constructed in a compact size.

Next, a process for manufacturing the optical DNA sensor will be described below.

First, a plurality of solid imaging devices 2 are simultaneously manufactured on one piece of transparent substrate. A process for manufacturing one of the solid imaging devices 2 is as follows.

Namely, a conductive layer is formed onto a transparent substrate 17 according to the PVD method or the CVD method, such as sputtering or vapor deposition. Then, a masking process, such as the photolithography method, and a form manufacturing process for manufacturing the form of the conductive substance layer by etching or the like are performed to thereby make patterning of the bottom gate electrode 41 and bottom gate lines 41, 41, . . . in each of the sensor 20.

In the next place, a bottom gate insulated film 22 comprising silicon nitride or silicon oxide is formed substantially throughout on the surface of the transparent substrate 17, a layer of semiconductor that becomes the semiconductor layer 23 is further formed throughout the surface of the bottom gate insulated film 22, and an insulated layer comprising silicon nitride or silicon oxide that becomes the channel protective film 24 is formed throughout the surface of the semiconductor layer. Then, the insulated layer is masked, and the form of the insulated layer is manufactured to pattern the channel protective film 24 for every sensor 20. Following thereto, an amorphous silicon layer containing n-type impurities is formed. Then, this amorphous silicon layer is masked, and the form thereof is manufactured to thereby make patterning of the impurities semiconductor layers 25, 26 as well as patterning of the semiconductor layer 23 placed underneath them for every sensor 20.

Next, the conductive layer is formed over the whole surface, the conductive layer is masked and the form thereof is manufactured to thereby make patterning of a drain electrode 28 and a source electrode 27 for every sensor 20 as well as patterning of data lines 43, 43, . . . and source lines 42, 42, . . .

Then, a top gate insulated film 29 is formed over the whole surface of the bottom gate insulated film 22 in which the drain electrode 28, the source electrode 27 and the like are formed. Next, a transparent conductive substance layer such as ITO is formed over the whole surface of the top gate insulated film 29, the transparent conductive substance layer is masked and the form thereof is manufactured to thereby make patterning of the top gate electrode 30 for every sensor 20 while simultaneously forming the top gate lines 44, 44, . . . in one united body with the top gate electrode 30.

Next, a protective insulated layer 31 is formed over the whole surface of the bottom gate insulated film 22 on which the top gate electrode 30 and the top gate line 44 are formed. Then, a conductive member layer 32 is formed over the whole surface of the protective insulated layer 31.

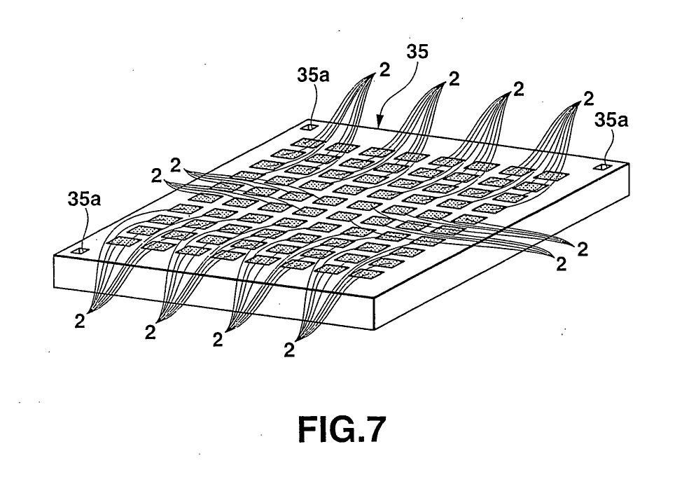

By simultaneously carrying out the above-described processes for the solid imaging device 2, a plurality of the solid imaging devices 2, 2, . . . are simultaneously manufactured on one sheet of transparent substrate 17, as shown in FIG. 7. In the following, a sheet of transparent substrate on which a plurality of solid imaging devices are manufactured is referred to as a mother substrate.

Next, at least one of the four corners of the mother substrate that is located on the surface (the conductive member layer 32) of the mother substrate is marked. In FIG. 7, marks are applied to three corners of the mother substrate. Then, the surface of the mother substrate 35 is chemically applied to form an overcoat layer 33 comprising, for example, polycation (poly-L-lysine, poly(ethylene imine) and the like) or a silane coupler on the surface of the mother substrate 35.

On the other hand, a plurality of DNA segments 61 each including a known nucleotide sequence are formed, (the nucleotide sequences of the respective types of DNA segments are different from one another), and the respective types of DNA segments are dispersed or dissolved in a solvent separately to prepare a plurality types of sample solutions. The plurality types of sample solutions are then separately put into a plurality of pipettes in a dispenser. Besides, the mother substrate 35 is set up on a setting table of the dispenser. This dispenser is configured so as to control the plurality of pipettes to move in a horizontal plane over the setting table and descend to apply the sample solution in a droplet.

Next, while impressing a positive voltage to the conductive member layer 32 formed on the surface layer of the mother substrate 35, the plurality types of sample solutions are respectively applied in a droplet onto the mother substrate 35 by means of a dispenser. At this time, various types of sample solutions are assigned to the respective solid imaging devices such that each of the solid imaging devices 2 is applied with a drop of different type of sample solution. The application of the sample solutions is carried out such that one type of sample solution is superimposed on the sensor 20 when it is observed in the plan view. A nucleotide strand comprising four bases of adenine, guanine, cytosine and thymine is negatively charged as a whole since a sugar bonding to a base is connected by a phosphodiester bond. As a result, with the positive voltage impressed to the conductive member layer 32, the DNA probe 61 are attracted. Therefore, the DNA probe 61 tend to be easily bonded to the overcoat layer 33 thanks to electrostatic coupling. Note that it is possible to read the marks 35a on the mother substrate 35 by means of the dispenser to adjust the droplet application positions, whereby applying the sample solution in a droplet onto each of the sensors 20 with good positional accuracy.

Then, the mother substrate 35 is cut for every solid imaging device 2 to thereby complete a plurality of optical DNA sensors 1.

Now, a DNA identification method using the optical DNA sensor 1 and the DNA reading apparatus 70 will be described below.

First, DNA is collected from a sample, and the collected DNA is denatured to a single strand DNA segment. Then, a fluorescent substance or a photoresonance scattering substance is bonded to the DNA segment to label the DNA segment with the fluorescent substance or the photoresonance scattering substance. As the fluorescent substance, Cy 2 (manufactured by Amasham Corp.) of CyDye is used, for example. The obtained DNA segment is contained in the solution. In the following, this DNA segment is referred to as “sample DNA segment”. For the fluorescent substance and the photoresonance scattering substance, a substance that is excited by a wavelength of the phosphor exciting light projected from the light irradiation means 70 in the DNA reading apparatus 70 should be selected. The fluorescent substance or the photoresonance scattering substance absorbs the phosphor exciting light to be excited, thereby emitting visible light. However, it is desirable for the wavelength range of the phosphor exciting light to be different as much as possible from the wavelength range of visible light that excites the semiconductor layer 23, and the wavelength range of the visible light is desirably in a range where it causes the semiconductor layer 23 of the optical DNA sensor 1 to generate sufficient charges.

When the optical DNA sensor 1 is attached to the DNA reading apparatus 70, the top gate lines 44, 44, are respectively connected to the terminals of the top gate driver 11, the bottom gate lines 41, 41, . . . are respectively connected to the terminals of the bottom gate driver 12, and the data lines 43, 43, . . . are respectively connected to the terminals of the data driver 13.

Next, a solution containing the sample DNA segment is coated onto a surface of the optical DNA sensor 1. The sample DNA segment hybridize to a complementary DNA probe 61 selected from among the spots 60, 60, . . . but does not bind to a DNA segment that is not complementary. From among the sample DNA segments coated to the optical DNA sensor 1, the segments which were not involved with the hybridization are washed out.

Following to the above, the light irradiation means 71 is turned on, and, when the phosphor exciting light like a plane of light is irradiated to the surface of the optical DNA sensor 1, the DNA reading apparatus starts reading. The image acquisition operation of the DNA reading apparatus is as follows. Here, explanation is given in detail on the operation of the sensor 20 at the i-th line.

First, during a period of resetting the i-th line, when the top gate driver 11 has impressed a positive reset voltage from the driving circuit 10 to the top gate line 44 of the i-th line in accordance with a control signal Tcnt, a positive voltage is relatively impressed to the top gate electrode 30 in the sensors 20, 20, . . . of the prefixed line within an image reading circuit 2 to release holes accumulated in the semiconductor later 23 and the channel protective film 24.

The spot 60, where the DNA probe 61 bonded with the complementary sample DNA segment by hybridization exists, receives phosphor exciting light. that is irradiated by the fluorescent substance from the light irradiation means 71 to emit visible light with a longer wavelength. Accordingly, the semiconductor layer 23 in the sensor 20 directly underneath the spot 60 is excited by the visible light to produce a number of electron-hole pairs. During the charge storage period of the i-th line following to the reset period, the top gate driver 11 impresses a negative charge storage voltage to the top gate line 44 of the i-th line, only the holes of positive charges are trapped by the semiconductor layer 23 and the channel protective film 24 thanks to negative electric field impressed to the top gate electrode 30, and the electrons are caused to repel against the electric field and result in discharging out of the sensor 20.

In the spot 60 where the DNA probe 61 that did not bind to the complementary sample DNA segment exists, visible light is not emitted in response to phosphor exciting light irradiated from the light irradiation means 71 during the charge storage period. As a result, almost no electron-hole pair is produced in the semiconductor layer 23 of the sensor 20 directly underneath the spot 60. Due to this, even though a charge storage voltage is impressed to the top gate electrode 30 after resetting, the holes are not stored in the semiconductor layer 23 and the channel protective film 24. Following to the above, during the pre-charging period of the i-th line, the data driver 13 outputs a pre-charge voltage at a high level to all of the data lines 43, 43, . . . and causes the drain electrode 28 to be maintained at +10 [V] via the data lines 43, 43, . . .

Then, after the top gate electrode 30 is impressed with a voltage of −20 [V], and during the reading period of the i-th line after the time at which the holes having been continuously stored in the semiconductor layer 23 of the sensor 20 directly underneath the spot 60, in which the DNA probe 61 bonded with the complementary sample DNA segment exists, have reached to a sufficient quantity, the bottom gate driver 12 impresses a voltage of +10 [V] to the bottom gate electrode 21. Where, since sufficient light cannot be incident in the sensor 20 directly underneath the spot 60 where no DNA probe 61 bonded with the complementary sample DNA segment exists, the holes have not been stored in the semiconductor layer 23 and the channel protective film 24. As a result, in the semiconductor layer, the electric field for forming channels generated by the voltage of +10 [V] from the bottom gate electrode 21 is counteracted by the electric field for disappearing channels generated by a voltage of −20 [V] from the top gate electrode 30, resulting in empty layers expanding into the semiconductor layer. Accordingly, currents are not transmitted to between the source and drain electrodes, and the pre-charge voltage of the data line 43 is retained as it is.

In the sensor 20 directly underneath the spot 60 in which the DNA probe 61 bonded with the complementary sample DNA segment exists, the holes are stored in the semiconductor layer 23 and the channel protective film 24. Although the holes have been affected by the electric field of −20 (V) so that they are attracted to the top gate electrode 30, but they have at the same time-a function to offset the negative electric field of the top gate electrode 30 with the charge amount of the holes. From this reason, the channel is not formed when the bottom gate electrode 21 has 0 (V). However, when the bottom gate electrode 21 has changed to have +1 (V), the electric field of the bottom gate electrode 21 and the positive electric field generated by the stored holes become stronger than the negative electric field of the top gate electrode 30 to thereby form a channel in the semiconductor layer 23. Accordingly, a current flows from the drain electrode 28 of which potential becomes high thanks to the pre-charge voltage to the source electrode 27 connected to the earth, and the potential of the data line 43 comes to a low level.

During the reading period, the charges stored during the charge storage period as described above work to relax the voltage between the top gate electrode 30 and the bottom gate electrode 21. As a result, a channel is formed in the semiconductor layer 23 thanks to the voltage between the bottom gate electrode 21 and the top gate electrode 30, whereby a current is allowed to flow from the drain electrode 28 to the source electrode 27. Therefore, during the reading period, there is a tendency that the voltages of the data lines 43, 43, . . . gradually drop in association with the time elapse under the influence of the current between the drain and source electrodes.

As the amount of fluorescence light having been incident to the semiconductor layer 23 increases during the charge storage period, the amount of the stored charges increases. As the amount of the stored charges increases, the level of the current flowing from the drain electrode 28 to the source electrode 27 increases during the reading period. Therefore, the tendency in the voltage changes of the data lines 43, 43, . . . during the reading period is deeply associated with the intensity and irradiation period of time of light emitted by the fluorescent substance that was incident to the semiconductor layer 23 during the charge storage period. Then, during a period from the reading period for the i-th line to the pre-charging period for the (i+1)-th line, the driving circuit 10 detects voltages of the data lines 43, 43, . . . via the data driver 13 after a prefixed time elapse following to the start of the reading period. Then, the detected voltages are converted to intensities of light. Note that, during a period from the reading period for the i-th line to the pre-charging period for the (i+1)-th line, the driving circuit 10 may be configured to detect a period of time required to reach to a prefixed voltage via the data driver 13. In this case as well, the detected voltages are converted to intensities of light.

As described above, fluorescence (mainly visible light) is emitted from the fluorescent substance bound to the sample DNA segment in the pairs of the DNA probe 61 in the spots 60, 60, . . . and the sample DNA segment bonded with the DNA probe 61, while no fluorescence is emitted from the DNA probe 61 having not bonded with the sample DNA segment. Accordingly, fluorescence with high intensity is incident to the sensor 20 corresponding to the spot 60 that includes the DNA probe 61 having bonded with the sample DNA segment, while almost no fluorescence is incident to the sensor 20 corresponding to the spot 60 comprising the DNA probe being not bonded with the sample DNA segment. Since the DNA probe 61 in the spots 60, 60, . . . are fixed onto the surface of the solid imaging device 2, fluorescence emitted from the spot 60 having bonded with the sample DNA segment is not attenuated so much and is incident to the sensor 20 corresponding to the spot 60 to generate electron-hole pairs. Therefore, the intensity of the fluorescence can be sensed sufficiently even though the sensitivity of the sensors 20, 20, . . . . is low. Besides, when a photoresonance scattering substance is bonded to the sample DNA segment, the spots having bonded with the sample DNA-segment from among the spots 60, 60, . . . are caused by resonance to emit light with high intensity, while the spots being not bonded with the sample DNA segment emit light with low intensity.

As shown in FIG. 8, the timing of rising of the reset voltage at the (i+1)-th line of the top gate driver 11 is after falling of the reading voltage at the i-th line of the bottom gate driver 12. However, the timing of rising of the rest voltage at the (i+1)-th line of the top gate driver 11 is not limited to the above, and it may be in between from immediately after falling of the reset voltage at the i-th line of the top gate driver 11 and falling of the reading voltage at the i-th line of the bottom gate driver 12. Note that an outputting of the pre-charge voltage having outputted to the data lines 43, 43, . . . for the sensor 20 at the (i+1)-th line is set so as to be done after falling of the reading voltage at the i-th line of the bottom gate driver 12. Besides, the sensitivity of the sensor 20 of the optical DNA sensor 1 can be controlled by adjusting the duration of the charge storage period. For example, when the charge storage period is prolonged, the period of time of the electron-hole pairs to be generated becomes longer even though the intensity of light emitted from the spot 60 having been subjected to hybridization is weak. Accordingly, the amount of the holes to be stored is increased so that light of the hybridized spot 60 can be sensed.

By repeating procedures similar to a series of the image reading operations as described above as one cycle for each of the sensors 20 in all lines, a distribution in the intensities of light on the optical DNA sensor 1 is obtained in the form of image data. The data driver 13 reads the potential drop in the data lines 43, 43, in which presence or absence of the incidence of visible light emitted by the fluorescent substance, that is bound to the sample DNA hybridized, results in the difference, and outputs the read potential drop to the driving circuit 10. The operation processor 4 can confirm presence of a base having a complementary nucleotide sequence to the probe DNA in the sample DNA based on the data on the potential drop inputted from the driving circuit 10 and also reads the position of the sensor 20 where hybridization has occurred. The operation processor 4 also stores nucleotide sequences of the probe DNA for each of the spots 60, 60, . . . , measures the position of the sensor 20 described hereinabove to calculate the position of the spot 60 over the sensor 29 to thereby determine the nucleotide sequence of the spot 60, deduces the nucleotide sequence of the complementary sample DNA automatically, and displays the nucleotide sequence of the identified sample DNA on a display 3.

The DNA reading apparatus 70 causes the optical DNA sensor 1 to drive to sense intensity of fluorescence or quantity of fluorescent light with each sensors and acquires light intensity distribution on the optical DNA sensor 1 in the form of image data of two dimensions. Since the distance between two adjacent sensors 20, 20 is at least 10 μm, the distance from the semiconductor layer 23 of the sensor 20 to a pair of DNA segments is 6,000 nm more or less, and the linear distance of the helix of a pair of DNA segments is 340 nm more or less even tough 1,000 bases are arrayed respectively in the DNA probe 61 and in a pair of sample DNA segments, fluorescence from the pair of DNA segments never be incident to a sensor 20 which is adjacent to the closest sensor to the pair of DNA segments to an extent to sufficiently produce the electron-hole pairs, irrespective of that the DNA probe 61 and a pair of sample DNA segments stand on the surface of the solid imaging device 2 or lay down thereon. In other words, since the sensors 20, 20, . . . are sufficiently remote from one to another, even tough the spots 60, 60, . . . are arranged so as to correspond to the sensors 20, 20, . . . , respectively, a pair of DNA segments never emit fluorescence of such a extent that it can sufficiently excite the neighbor sensor 20, if the length of the DNA segment is 1,000 nm or less. In addition, when each sensor 20 is caused to correspond to each spot 60, many types of nucleotide sequence, the numbers thereof is equivalent to the numbers of the sensors 20, can be identified at once.

### Second Embodiment

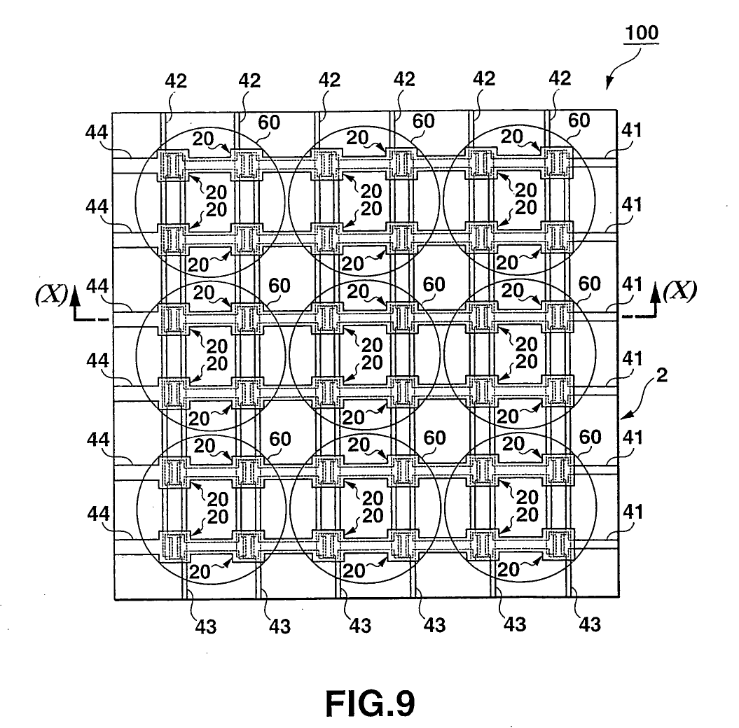

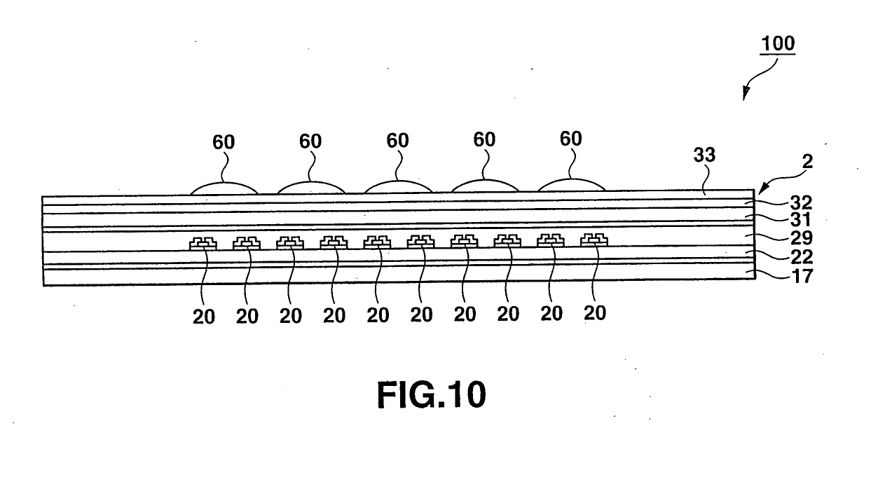

FIG. 9 is a plan view showing an optical DNA sensor according to the second embodiment, and FIG. 10 is a cross-section of the optical DNA sensor when it is cut along a broken line (X)-(X) indicated in FIG. 9 and observed to a direction indicated by arrows.

In the optical DNA sensor 1 according to the first embodiment, one sensor 20 corresponds to one spot 60. Unlike that, in the optical DNA sensor 100 according to the second embodiment, sensors 20 are fixed on the surface of a solid imaging device such that four sensors 20 correspond to one spot 60. Specifically, in the optical DNA sensor according to the second embodiment, four adjacent sensors 20 in the vertical and horizontal directions form a set, and one spot corresponds to the set. When observing in a plan view, four sensors 20 are superimposed on one spot 60. Note that the neighbor spots 60 are distanced to each other.

The other components of the optical DNA sensor of this embodiment are equivalent to those of the optical DNA sensor according to the first embodiment. Therefore, detailed explanation on the other components of the optical DNA sensor 100 is omitted. Like the optical DNA sensor 1, the optical DNA sensor 100 of this embodiment can be used for the DNA reading apparatus, where the optical DNA sensors of this embodiment can be used in the DNA identification method in the same procedures as those described in the first embodiment except that light emitted from one spot 60 is received by the corresponding four sensors 20. Furthermore, the manufacturing process of the optical DNA sensor 100 is same as the process for the one of the first embodiment except that one spot 60 is fixed for four sensors 20.

Besides, in the manufacturing process for the optical DNA sensor 100, the number of sensors 20 is not limited to four. It may be configured such that one spot 60 corresponds to two or three adjoining sensors in either a longitudinal or lateral direction, or one spot 60 corresponds to five or more adjoining sensors 20. However, it should be configured that any one of the spots 60 in a plane correspond to the same number of sensors. In any case, when the number of sensors 20 corresponding to one spot 60 is represented by A (A is an integer of 2 or more), and the number of the spots 60 is represented by B, the number represented by (A×B) is the minimum number of the required sensors 20 to be contained in the solid imaging device 2. In order to avoid different DNA probe 61 from mixing to each other under a condition that the adjacent spots 60 are too close to contact with each other with just a slight swing, the optical DNA sensor may be configured such that a sensor 20, on the upper surface of which no spot 60 is positioned, is interposed between two adjacent spots 60, respectively, and the optical DNA sensor 100 may be provided with more than (A×B) pieces of sensors 20.

In this embodiment, like the first embodiment, since the spots 60, 60, . . . are arrayed and fixed on the surface of the solid imaging device 2, it is needless to provide the DNA reading apparatus 70 with optical systems, such as lenses and microscopes. Accordingly, it is possible to construct the DNA reading apparatus in a compact size.

Besides, when light emitted from the spot 60 having bonded to the sample DNA segment is weak, there is a fear that the light with such a weak intensity cannot be sufficiently sensed by just one sensor 20. However, since more than 2 sensors 20 correspond to one spot 60, the light emitted from one spot 60 is received by two or more sensors 20. Therefore, light even with weak intensity can be sensed securely. In this concern, it may be configured such that the optical information data calculated by all of a plurality of sensors 20 corresponding to one spot 60 is added to establish a criterion of nucleotide sequence identification, or such that the optical information data of only one sensor 20 that sensed the strongest quantity of light from among the plurality of sensors 20 corresponding to one spot 60 is used as the criterion of nucleotide sequence identification. Besides, there is also a case where, due to presence of a sensor 20 with a failure in between the source and drain electrodes or the like, a drain current is happened to flow even though no fluorescence has actually been emitted, and the voltage of the data line 43 during the reading period accordingly falls to thereby allow the sensor 20 to misidentify presence of fluorescence emission. For keeping up with such a case, it may be configured such that optical information data of a sensor 20 that has sensed the maximum quantity of light from among the plurality of sensors 20 corresponding to one spot 60 is excluded, and the nucleotide sequence identification is performed from a criterion that bases on the remaining optical information data of the other sensors 20. Similarly, there might be a case where, due to presence of a sensor 20 with a failure in between the source and drain electrodes or the like, a drain current does not flow even though fluorescence has actually been emitted, and therefore the voltage of the data line 43 during the reading period does not fall to thereby allow the sensor 20 to misidentify no presence of fluorescence emission. For keeping up with such an misidentification, it may be configured such that optical information data of a sensor 20 that has sensed the minimum quantity of light from among the plurality of sensors 20 corresponding to one spot 60 is excluded, and the nucleotide sequence identification is performed from a criterion that bases on the remaining optical information data of the other sensors 20. Further, taking the above into consideration, the nucleotide sequence identification may be performed on the basis of the optical information data of the plurality of sensors 20 corresponding to one spot 60, from which the optical information data of a sensor 20 having sensed the maximum quantity of light and a sensor 20 having sensed the minimum quantity of light from among the plurality of sensors 20 are excluded. With such a configuration as described above, the identification of one type of nucleotide sequence is compensated with a plurality of sensors 20. Therefore, even though a sensor 20 from among the plurality of sensors has a failure, optical information data can be employed from the remaining sensors 20 capable of operating normally. Accordingly, the nucleotide sequence can be read with accuracy.

### Third Embodiment

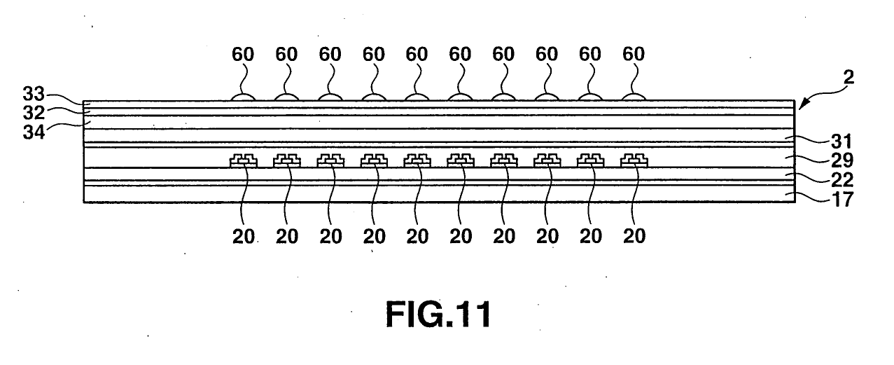

As shown in FIG. 11, the optical DNA sensor according to the third embodiment is structured by additionally including an excited light absorbing layer 34 to the optical DNA sensor according to either of the above-described embodiments. FIG. 11 is a cross-section of the optical DNA sensor of the third embodiment which is similar to the sensor of the first embodiment shown in FIG. 3.

The optical DNA sensor 1 of this embodiment includes a solid imaging device 2, an excited light absorbing layer 34 formed on the surface of the solid imaging device and made from a titanium oxide layer with a fixed thickness, and spots 60, 60, . . . arrayed and fixed on the excited light absorbing layer 34, wherein each of the spots 60 corresponds to each of pixels of the solid imaging device.

The solid imaging device 2 includes a transparent substrate 17 in a substantially flat plate shape and sensors 20, 20, . . . each having a plurality of double gate type field-effect transistors arrayed in a matrix fashion consisting of n lines and m rows, (n and m are respectively an integer), on the surface of the transparent substrate 17.

The transparent substrate 17 has light-transmitting and insulating properties and is a substrate made of glass, such as quartz glass or plastic, such as polycarbonate. The reverse surface of the transparent substrate 17 constitutes the reverse surface of the solid imaging device 2. Note that a substrate having shading property may be used instead of the transparent substrate 17 having light-transmitting property.

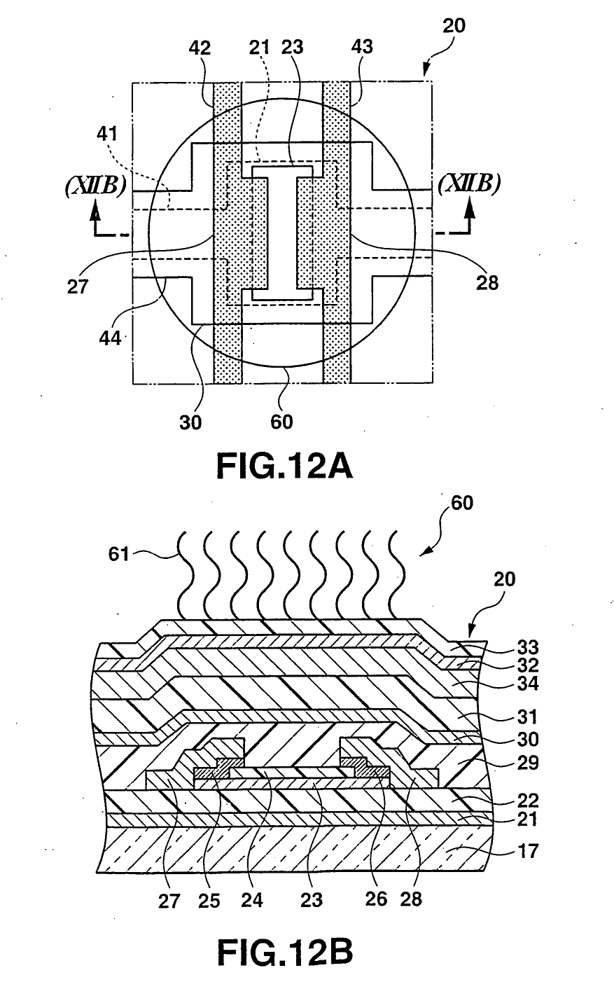

FIG. 12A is a plan view showing a sensor 20, and FIG. 12B is a cross-section of the sensor when it is cut along a broken line (XIIB)-(XIIB) in FIG. 12A and is observed to the direction indicated by arrows. Each of the sensors 20 is a photoelectric conversion element functioning as a pixel similar to the one in the first embodiment described above.

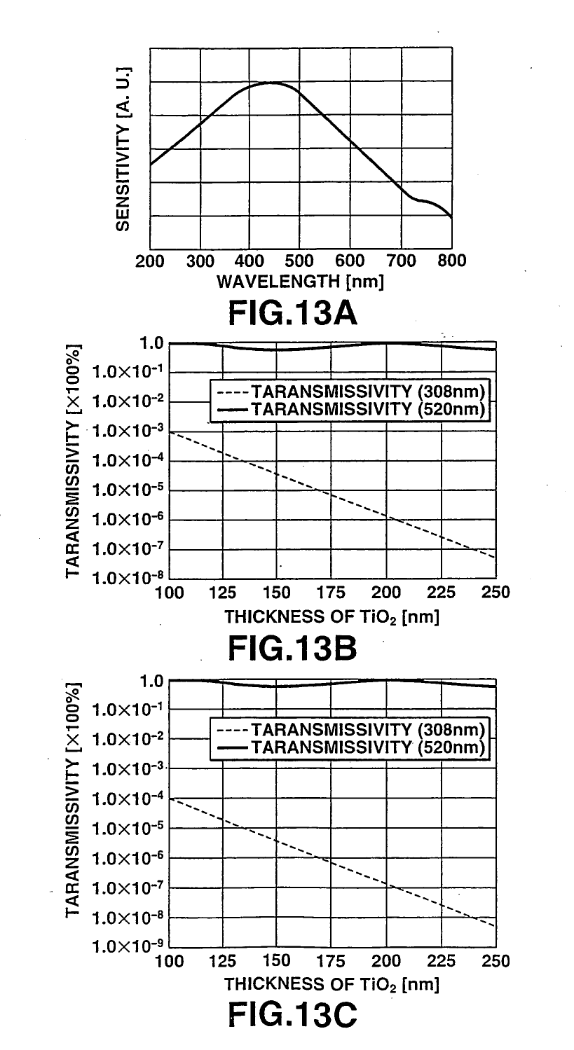

A semiconductor layer 23 is formed on a bottom gate insulated film 22 for each of the sensor 20. The semiconductor layer 23 has a substantially rectangular shape when it is observed in the plan view and is a layer made from amorphous silicon or polysilicon. A channel protective film 24 is formed on the semiconductor layer 23. The channel protective film 24 has a function to protect the interface of the semiconductor layer 23 from an etchant used for patterning, insulating and light-transmitting properties, and is made from, for example, silicon nitride or silicon oxide. The semiconductor layer 3 is sensitive to light, and, when light is incident to the semiconductor layer 23, it generates electron-hole pairs of a quantity in accordance with quantity of light having been incident to around the vicinity of the interface of the channel protective film 24 and the semiconductor layer 23. In this case, holes are generated as charges in the semiconductor layer 23 side, and electrons are generated in the channel protective film 24 side. Now, wavelength dependence of light sensitivity of amorphous silicon that has a thickness of 50 nm and is applicable for the semiconductor layer 23 is shown in FIG. 13A. The amorphous silicon has sensitivity with which electron-hole pairs are generated over a wide range of from ultraviolet rays to visible rays, and it shows a peak of sensitivity against visible light of around 450 nm.

On the surface of the solid imaging device 2, a protective insulated film 31, an excited light absorbing layer 34, a conductive layer and an overcoat layer are laminated in this order. The protective insulated film 31 coats all the sensors 20, 20, . . . in the block, and is formed over the top gate electrode 30 and the top gate lines 44, 44, . . . so as to coat them. The protective insulated layer 31 has insulating and light-transmitting properties and is made from silicon nitride or silicon oxide.

Over the protective insulated layer 31, the excited light absorbing layer 34 is formed so as to coat all the sensors 20, 20, . . . Titanium oxide contained in the excited light absorbing layer 34 is classified into the anatase-type and the rutile-type. Although both types can be used in the present invention, it is preferable to use the rutile-type. The crystalline structure of the rutile-type titanium oxide is tetragonal, and the arrangement of Ti is body-centered cubic structure.

The excited light absorbing layer 34 has a property to absorb a phosphor exciting light (mainly an ultraviolet ray particularly in a zone of around the central wavelength of 308 nm) that excites a fluorescent substance used for a DNA identification method described later and to transmit fluorescence emitted from the fluorescent substance excited by the phosphor exciting light (mainly visible light particularly in a zone of around the central wavelength of 520 nm).

An extinction coefficient k (>0), that is an optical solid-state parameter for characterizing absorption, has a relation represented by the following equation (1) between itself and a complex index of refraction N.
N=n−ik (1)

In the equation (1), i is an imaginary unit, n determines the phase velocity of waves of light heading for a given direction, and the extinction coefficient k has a function to attenuate the magnitude of light wave amplitudes together with a heading direction of light waves. When the heading direction of light is represented by z and the intensity of light is represented by I, the following equation (2) is given for the relation between the two.
I(z)=I(O)c×p(−αz) (2)

In the above equation, α is an absorption coefficient and is expressed by the following equation.
α=2ωk/c (3)
c is a velocity of light in a vacuum, and ω is an angular velocity of light.

The excited light absorbing layer 34 of the rutile-type crystal is cubic, and, considering the configuration of a titanium atom, it has a body-centered cubic structure. This crystal is a uniaxial crystal of which optical axis exists in C axis. Although the complex index of refraction N accurately differs depending on an angle between an electric field vector of an incident light and the C axis, the extinction coefficient k of a ultraviolet ray of 300 nm more or less is 2 in average, and the extinction coefficient k of a visible ray of 440 nm more or less comes to 0.06. In case of a visible ray of 460 nm, the extinction coefficient k can be assumed as k=0.

In FIG. 13B, a relation between the thickness of the excited light absorbing layer 34 and transmittances of the phosphor exciting light with a wavelength of 308 nm and fluorescence with a wavelength of 530 nm is shown in a logarithmic graph. As shown in FIG. 13B, as the thickness of the excited light absorbing layer 34 increases, the transmittance of the phosphor exciting light is lowered. When the thickness of the excited light absorbing layer 34 is 100 nm or greater, the transmittance of the phosphor exciting light becomes 1.0×10⁻³ or less. On the other hand, the transmittance of the fluorescence is not low as much as that of the phosphor exciting light and is 50% or more irrespective of the thickness of the excited light absorbing layer 34.

As shown in FIGS. 11 and 12, a conductive layer 32 is formed over the excited light absorbing layer 34. The conductive layer 32 has conductive and light-transmitting properties and is made from, for example, indium oxide, zinc oxide or tin oxide, or a mixture comprising at least one thereof. The excited light absorbing layer 34 absorbs the phosphor exciting light to produce the electron-hole pairs. Although a part of the pairs remains in a state of no recombination, charges caused by the electron-hole pairs are discharged by the conductive layer 32 since the conductive layer 32 is contact with the excited light absorbing layer 34. Accordingly, the electrons and holes are never continuously stored in the excited light absorbing layer 34 and the protective insulated layer 31. Therefore, there is almost no influence to the electric field formed by a voltage to be impressed to the top gate electrode 30.

Throughout on the conductive layer 32, an overcoat layer 33 is formed. This overcoat layer 33 has light-transmitting property and works to protect the conductive layer 32 and fix the spots 60, 60, . . . on the surface of the solid imaging device 2.

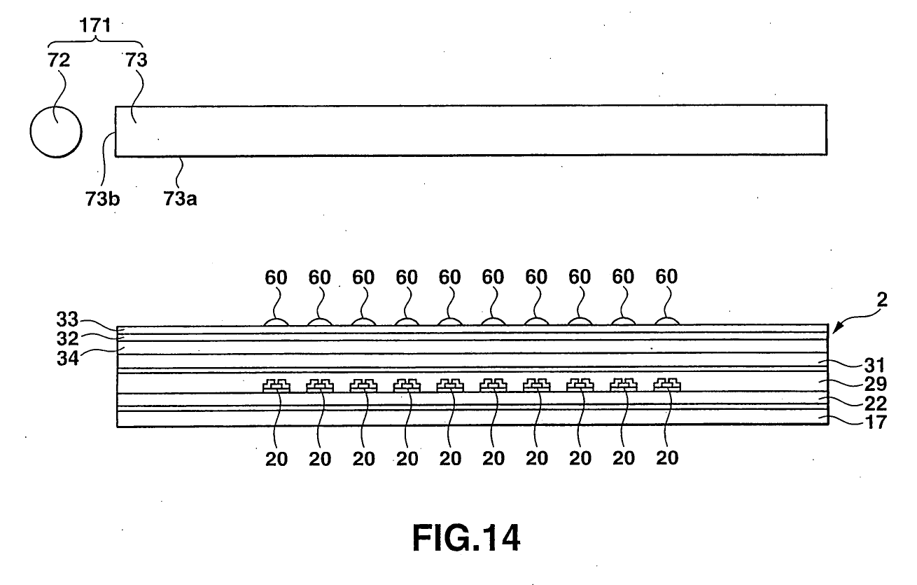

Now, explanation is given on the DNA reading apparatus using the optical DNA sensors constituted as described above with reference to FIGS. 5 and 14.

As shown in FIGS. 5 and 14, the DNA reading apparatus 70 includes a display 3, an operation processor 4 for controlling the whole apparatus, a light irradiation means 74 for irradiating the phosphor exciting light in a state like a plane of light onto the surface of the optical DNA sensor 1 and a driving means (comprising a top gate driver 11, a bottom gate driver 12, a data driver 13 and a driving circuit 10) for driving the optical DNA sensor to acquire images.

The light irradiation means 74 includes a light source for emitting light that include a wavelength range of the phosphor exciting light but does not include a wavelength range of the fluorescence so much, and a light-guiding plate 73 for guiding light emitted from the light source 72 therethrough to project the light in a state like a plane of light from the reverse surface 73a of the light-guiding plate. The light-guiding plate 73 has a substantially flat plate shape and is coated with a reflective member except the side 73b facing to the light source 72 and the reverse surface 73a. The optical DNA sensor 1 is constituted such that the sensor can be attached to and removed from the DNA reading apparatus 70 and the surface of the solid imaging device 2 of the optical DNA sensor 1 attached to the DNA reading apparatus 70 faces to the reverse surface 73a of the conductive layer 73. The optical DNA sensor 1 is constituted such that light in a state like a plane of light projected from the reverse surface 73a of the light-guiding plate 73 is uniformly irradiated to the surface of the optical DNA sensor 1 when the optical DNA sensor 1 has faced to the reverse surface 73a of the light-guiding plate 73.

Further, it is configured such that, when the optical DNA sensor 1 is attached to the DNA reading apparatus 70, the top gate lines 44, 44, . . . of the optical DNA sensor 1 are respectively connected to the terminals of the top gate driver 11. Similarly, it is configured under the same situation such that the bottom gate lines 41, 41, . . . of the optical DNA sensor 1 are respectively connected to the terminals of the bottom gate driver 12, and the data lines 43, 43, . . . of the optical DNA sensor 1 are respectively connected to the terminals of the data driver 13. Further, it is configured such that, when the optical DNA sensor 1 is attached to the DNA reading apparatus 70, the source lines 42, 42, . . . of the optical DNA sensor 1 are connected to a given voltage source or to the earth in this embodiment.

Since the spots 60, 60, . . . are arrayed on the surface of the solid imaging device 2, clear images can be imaged by means of the solid imaging device 2 without proving the DNA reading apparatus 70 with optical systems, such as lenses and microscopes. Accordingly, the DNA reading apparatus can be constructed in a compact size.

Now, explanation is given on the process for manufacturing the optical DNA sensor 1.

The manufacturing process for the optical DNA sensor 1 according to the third embodiment is same as the process described in the first embodiment up to the step where the protective insulated layer 31 is formed.

Following to that step, an excited light absorbing layer 34 is formed in a film state throughout on the protective insulated layer 31. Then, a conductive layer 32 is formed in a film state throughout on the excited light absorbing layer 34. Further, the conductive layer 32 is chemically-processed to form an overcoat layer 33, which is made from, for example, polycation (such as poly-L-lysine and poly(ethylene imine) or a silane coupler, in a film state on the conductive layer 32.

On the other hand, a plurality types of DNA segments including known nucleotide sequences are produced, (the nucleotide sequences in the plurality types of DNA segments are different from one to another), and the plurality types of DNA segments are dispersed or dissolved with a solvent to prepare a plurality types of sample solutions. The plurality types of sample solutions having been prepared are placed into a plurality of pipettes provided in a dispenser, respectively. Further, the solid imaging device 2 is set on a setting table provided to the dispenser. In this dispenser, the plurality of pipettes move on the setting table in the horizontal direction and further descend to apply the sample solution in a droplet.

Then, while keeping to impress a positive voltage to the conductive film 32, the plurality types of sample solutions are respectively applied in a droplet from the pipette to the surface of the solid imaging device 2 (onto the overcoat layer 33) by means of the dispenser. At this time, the one type of sample solution is applied in a droplet such that it is superimposed onto one sensor 20 when the sensor is observed in the plan view. A nucleotide sequence comprising four types of bases, that is, adenine, guanine, cytosine and thymine, is negatively charged as a whole, since a sugar has bonded with one of the bases thanks to phosphoric diester bond. As a result, the DNA probe 61 is attracted under influence of an electric field of a positive voltage impressed to the conductive film 32. Therefore, it becomes easy to fix the DNA probe 61 onto the overcoat layer 33.

When all of the procedures described above have been carried out, the optical DNA sensor 1 is completed.

The DNA identification method using the optical DNA sensor 1 and the DNA reading apparatus according to the third embodiment is same as the method described in the first embodiment. However, there are some differences in the operation therebetween. Now, an explanation is given below mainly on the difference.

A solution containing sample DNA segments obtained by sampling DNA from a test sample is coated onto the surface of the optical DNA sensor 1. The sample DNA segments bind to the complementary DNA probe 61 from among the spots 60, 60, . . . thanks to hybridization but do not bind to DNA probe that are not complementary. Of the sample DNA segments coated onto the optical DNA sensor 1, the segments which were not hybridized are washed out.

Then, the light source is turned on, and the phosphor exciting light is irradiated from the light-guiding plate 73 to throughout on the surface of the optical DNA sensor 1, and the DNA reading apparatus 70 starts to read in response to the irradiation of phosphor exciting light. Following to the irradiation, fluorescence is emitted from the fluorescent substance bound to the sample DNA segments in the spots 60 of the set of the DNA probe 61 and the sample DNA segments having bonded with the DNA probe 61, but no fluorescence is emitted in the spots 60 of the DNA probe that did not bind to the sample DNA segments. Fluorescence emitted from the spots 60 containing the DNA probe bonded with the sample DNA segments transmits the overcoat layer 33, the conductive layer 32, the excited light absorbing layer 34, the protective insulated layer 31, the top gate electrode 30, an interlayer insulated film 20 and the channel protective film 24 and is incident to the semiconductor layer 23 of the sensor 20 corresponding to the spots that have emitted the fluorescence. At that time, a part of the phosphor exciting light is not converted to fluorescence and is incident to the excited light absorbing layer 34 underneath the spot 60 in which the hybridization occurred. However, since the wavelength range of such a phosphor exciting light is short, it is absorbed into the excited light absorbing layer 34 and almost no phosphor exciting light reaches to the semiconductor layer 23. On the other hand, fluorescence is not incident to the semiconductor layer 23 of the sensor 20 that corresponds to the spot 60 comprising the DNA probe having not bonded with the sample DNA segments. As a result, the phosphor exciting light is incident to the excited light absorbing layer 34. However, since the phosphor exciting light is absorbed into the excited light absorbing layer 34, it does not reach to the semiconductor layer 23. Hence, the phosphor exciting light does not reach the semiconductor layers 23 of all sensors 20 irrespective of occurrence of the hybridization. Because of that, there is no case that the semiconductor layers 23 are excited when the phosphor exciting light emitted from the light source 72 is directly incident to the semiconductor layers 23, and that the electron-hole pairs in a quantity of causing sufficient drain current flow is produced in the semiconductor layers 23. Accordingly, substantially no holes are accumulated in the semiconductor layer 23 of the sensor 20 that corresponds to the spot 60 comprising the DNA probe 61 having not bonded with the sample DNA segments, and a large quantity of holes are accumulated in the semiconductor layer 23 of the sensor 20 that corresponds to the spot 60 comprising the DNA probe having bonded with the sample DNA segments.

Then, the DNA reading apparatus 70 drives the optical DNA sensor 1 to thereby render the optical DNA sensor to cause each sensors 20 to sense the intensity or quantity of light of the fluorescence and acquires the fluorescence intensity distribution on the optical DNA sensor 1 as image data of two dimensions.

As described above, in this embodiment, since the phosphor exciting light is absorbed and shaded by the excited light absorbing layer 34, substantially no phosphor exciting light is incident to the semiconductor layer 23. However, the fluorescence is not shaded and is incident to the semiconductor layer 23. As a result, only the semiconductor layer 23 of the sensor 20 that corresponds to the spot 60 having bonded with sample DNA segment produces the electron-hole pairs. Therefore, difference between the light intensity sensed by the sensor 20 that corresponds to the spot 60 having bonded with the sample DNA segment and the light intensity sensed by the sensor 20 that corresponds to the spot 60 being not bonded with the sample DNA segment becomes greater. As a result, contrast in images that represent the fluorescence intensity distribution is improved, the production of the electron-hole pairs as noise is inhibited even though the intensity of the phosphor exciting light is increased, and determination of nucleotide sequences in the sample DNA segments can be facilitated.

Note that, although the excited light absorbing layer 34 is laminated on the protective insulated layer 31 in the above description, the absorbing layer 34 may be laminated between the top gate insulated film 29 and the top gate electrode 30, or between the top gate electrode 30 and the protective insulated layer 31, or between the conductive layer 32 and the overcoat layer 33. Namely, the excited light absorbing layer 34 may be laminated in between any layers, as far as it is formed on the surface of the solid imaging device 2 and in a range between the semiconductor layer 23 and the spot 60.

Further, as the photoelectric conversion element, the solid imaging device 2 using the sensors 20, 20, is exemplified in the above description. However, instead thereof, a solid imaging device using photodiodes as the photoelectric conversion element may be used. Examples of the solid imaging device using photodiodes include a CCD image sensor and a CMOS image sensor.

In the CCD image sensor, photodiodes are arrayed in a matrix fashion on a substrate. In the circumference of each photodiode, a vertical CCD and a horizontal CCD adapted to transmit electric signals photoelectrically converted by the diode are formed. Further, similarly to the foresaid solid imaging device 2, a protective insulated film is formed throughout so as to coat a plurality of photodiodes, and a conductive film is formed throughout on the protective insulated film. A plurality types of spots are arrayed on the conductive film through an overcoat layer. When observing the photodiodes in the plan view, it is noted that one spot is superimposed on one photodiode.

In the CMOS image sensor, photodiodes are arrayed on a substrate in a matrix fashion. In the circumference of each photodiode, a pixel circuit adapted to amplitude electric signals photoelectrically converted by the photodiode is provided. Further, similarly to the solid imaging device 2, a protective insulated film is formed so that it coats throughout a plurality of photodiodes, and a conductive film is formed throughout on the protective insulated film. And, a plurality types of spots are arrayed on the conductive film via an overcoat film. When observing the spots in the plan view, one spot is superimposed on one photodiode.

Irrespective of using the CCD image sensor or CMOS image sensor, ultraviolet rays will not be incident to the photodiodes, if the excited light absorbing layer 34 is laminated between the spot and the photodiode and the photodiode is coated with the excited light absorbing layer 34.

### Fourth Embodiment

Now, the fourth embodiment for the present invention will be described below.

The difference of this embodiment from the optical DNA sensor according to the third embodiment exists in either the conductive layer 32 or the top gate electrode 30 of the optical DNA sensor 1. Also, in the fourth embodiment, an excited light absorbing layer 34 may be or may not be provided to the optical DNA sensor according to the fourth embodiment. Other constituents of the optical DNA sensor according to the fourth embodiment are same as those constituents of the optical DNA sensor 1 according to the third embodiment. With reference to FIGS. 1 to 12, the distinctive features of the optical DNA sensor of the fourth embodiment is explained in detail with use of the same reference numerals for the same constituents.

Namely, unlike the third embodiment wherein the excited light absorbing layer 34 absorbs the phosphor exciting light to shade it and has fluorescence-transmitting property, in the optical DNA sensor according to the fourth embodiment, at least one of the conductive layer 32 and top gate electrode 30 absorbs the phosphor exciting light to shade it and has fluorescence-transmitting property.

More specifically, the conductive layer 32 and the top gate electrode 30 are formed with ITO as well as those of the third embodiment. But, the charge density is controlled so as to be 1.0×10²⁰ [1/cm³] or less by controlling the film-forming speed, oxygen concentration in the atmosphere during the film formation and the like. That is to say, by adjusting the charge density of ITO to a level of 1.0×10²⁰ [1/cm³] or less, a threshold to separate wavelengths of light absorbable by ITO and wavelengths of light being not absorbable by ITO is shifted (Burstein-Moss shift) to absorb the phosphor exciting light but not to absorb the fluorescence. This process is achievable thanks to a change in the band gap caused by occupation of the bottom area of the conductive band by the charges produced by either oxygen failure of ITO or doped tin.

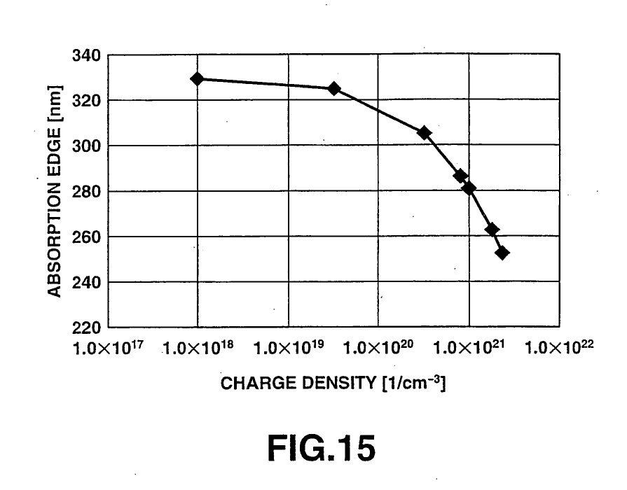

In FIG. 15, a relation between charge densities in ITO and absorption edge is shown. In FIG. 15, it is shown that light of a wavelength shorter than absorption edge is absorbed by ITO, and it is notable that the absorption edge shifts to longer wavelength side as the charge density of ITO lessens. Further, when the charge density of ITO exceeds 1.0×10²⁰ [1/cm³], the absorption edge comes to a low level, where the phosphor exciting light is not absorbed and is transmitted. However, when the charge density of ITO is 1.0×10²⁰ [1/cm³], the absorption edge comes to 308 nm, whereby ITO absorbs the phosphor exciting light with a wavelength of 308 nm or less. Further, when the charge density comes to 1.0×10¹⁹ [1/cm³], the absorption edge comes to 325 nm, whereby ITO absorbs the phosphor exciting light with a wavelength of 325 nm or less and transmits fluorescence.

As described above, the absorption edge of ITO of at least one of the conductive layer 32 and the top gate electrode 30 has been shifted to a greater energy side as the charge density increases. Therefore, by lessening the charge density, it enables ITO to absorb light of shorter wavelengths. In FIG. 13C, a relation of the thickness of the excited light absorbing layer 34 to the phosphor exciting light with a wavelength of 308 nm and the transmittance of fluorescence with a wavelength of 530 nm when the charge density of ITO of the conductive layer 32 or the top gate electrode 30 in the optical DNA sensor 1 with the configuration described in the third embodiment is set to 1.0×10¹⁹ [1/cm³] and the optical constant N of ITO is set to N (308 nm)=2.2−0.34i (wherein i is an imaginary unit). In comparison with FIG. 13B, where the charge densities of both conductive layer 32 and top gate electrode 30 exceed 1.0×1019 [1/cm3], it is noted that the phosphor exciting light with a wavelength of 308 nm is further shaded in the conductive layer 32 or the top gate electrode 30.

The manufacturing process for the optical DNA sensor according to the fourth embodiment is substantially same as that for the optical DNA sensor 1 according to the third embodiment, except that, when the ITO layer of the top gate electrode 30 and the conductive layer 32 are formed, the film-forming speed and oxygen concentration in the atmosphere are adjusted so that their charge densities come to a level of 1.0×10²⁰ [1/cm³] or less. Note that, when the film-forming speed is constant, it is possible to increase the partial pressure of oxygen in the ITO film-forming reactor to thereby reduce oxygen failure in the ITO as the oxygen concentration increases and the density of charges. In addition, when the partial pressure of oxygen in the ITO film-forming reactor, that is, oxygen concentration in the reactor atmosphere is constant, it is desirable that the density of charges can be reduced following to the reduction of the oxygen failure in the ITO in accordance with reduction of the film-forming speed, and the film-forming speed of the ITO can be reduced under a state of high partial pressure of oxygen.

As well as the optical DNA sensor 1 according to the third embodiment, the optical DNA sensor according to the fourth embodiment can be used in the DNA reading apparatus. In addition, the optical DNA sensor of this embodiment can be used for the DNA identification method in the same way as that of the third embodiment.

As described above, in the fourth embodiment, the phosphor exciting light is absorbed by the conductive layer 32 or the top gate electrode 30 and is then shaded, but fluorescence is not shaded and is incident to the semiconductor layer 23. Accordingly, only the semiconductor layer 23 of the sensor 20 corresponding to the spot 60 having bonded with the sample DNA segment is exposed. Therefore, contrast of images expressing fluorescence intensity distribution is improved, and determination of nucleotide sequences in the sample DNA segments can be facilitated.

Note that, even in the optical DNA sensor that uses the CCD image sensor or CMOS image sensor, the optical DNA sensor can be workable insofar as an ITO layer, of which charge density is 1.0×10²⁰ [1/cm³] or less, is laminated on the surface of the image sensor and the ITO layer is disposed between the spot and the photodiode.

### Fifth Embodiment

Now, the fifth embodiment for the present invention is described below.

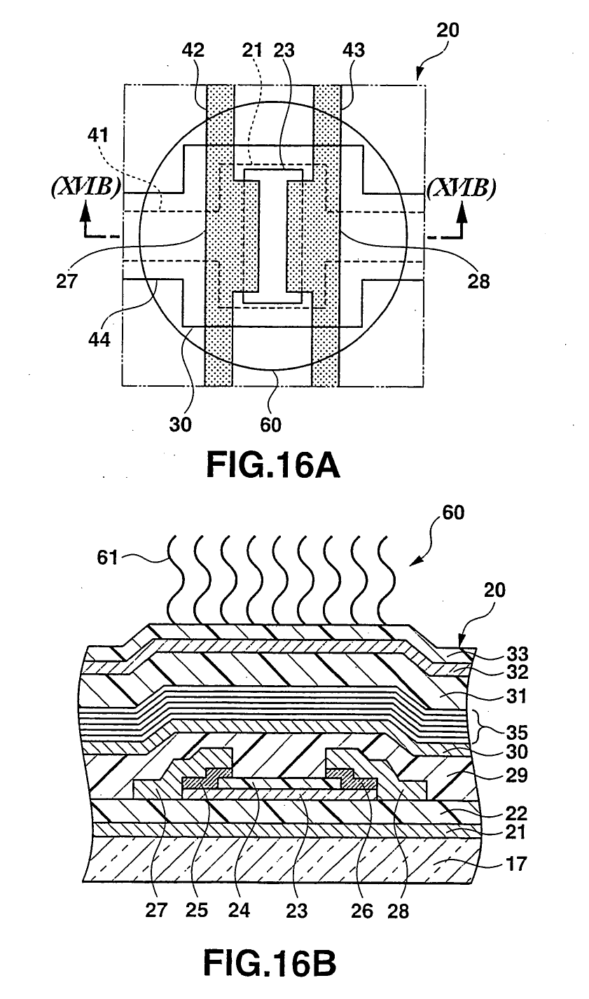

FIG. 16A is a plan view showing one pixel of the optical DNA sensor according to the fifth embodiment, and FIG. 16B is a cross-section of the pixel when it is cut along a broken line (XVIB)-(XVIB) indicated in FIG. 16A and is observed to the direction indicated by arrows.

Unlike that the excited light absorbing layer 34 is laminated between layers in the area extending from the semiconductor layer 23 to the spot 60 in the optical DNA sensor 1 according to the third embodiment, a dielectric multilayered film 35 is laminated between layers in the area extending from the semiconductor layer 23 to the spot 60 in the optical DNA sensor according to the fifth embodiment.

The dielectric multilayered film 35 has a multilayered structure, wherein a dielectric H layer of a high refractive index and a dielectric L layer of a refractive index lower than that of the dielectric H layer, the optical film thickness of each of those which is equivalent to one fourth of the central wavelength of the phosphor exciting light, are alternately laminated. When λ is a central wavelength of the phosphor exciting light and n₁ is a refractive index of the dielectric H layer, the film thickness of the dielectric H layer is represented as λ/4n₁, and when n₂ is a refractive index of the dielectric L layer, the film thickness of the dielectric L layer is represented as λ/4n₂. For example, using titanium oxide of a high refractive index (TiO₂; Refractive index 2.2) as the dielectric H layer and silicon oxide of a low refractive index (SiO₂; Refractive index 1.47) as the dielectric L layer, and laminating them alternately, a dielectric multilayered film 35 is completed. Reflection due to difference in refractive indexes occurs in the interfaces of each layers of the dielectric multilayered film 35, and the phosphor exciting light in a zone including the central wavelength interfere to each other. As a result, the phosphor exciting light comes to be reflected at an extremely high reflectance. On the other hand, the fluorescence is not reflected in the dielectric multilayered film 35 and transmits through it.

Note that the dielectric multilayered film 35 is not limited to the one prepared by alternately laminating two types of dielectric layers each having an optical film thickness equivalent to one fourth of the central wavelength of the phosphor exciting light, and the other film prepared by cyclically laminating three types of dielectric layers each having a different refractive index and an optical film thickness equivalent to one fourth of the central wavelength of the phosphor exciting light may also be used.

In FIG. 16, although the dielectric multilayered film 35 is laminated between the top gate electrode 30 and the protective insulated layer 31, the dielectric multilayered film 35 may be laminated between the protective insulated layer 31 and the conductive layer 32, or between the conductive layer 32 and the overcoat layer 33.

Like the optical DNA sensor 1 according to the third embodiment, the optical DNA sensor according to the fifth embodiment can be used for the DNA reading apparatus 70 and in the DNA identification method.

As described above, in the fifth embodiment, the phosphor exciting light is reflected by the dielectric multilayered film 35, but the fluorescence is not reflected and is incident to the semiconductor layer 23. Accordingly, only the semiconductor layer 23 of the sensor 20 corresponding to the spot 60 having bonded with the sample DNA segment is exposed. With such a manner, contrast of images expressing fluorescence intensity distribution is improved and determination of nucleotide sequences in the sample DNA segments can be facilitated.

Note that, even in the optical DNA sensor that uses the CCD image sensor or CMOS image sensor, the optical DNA sensor can be workable insofar as the dielectric multilayered film is laminated on the surface of the image sensor and the dielectric multilayered film is disposed between the spot and the photodiode.

### Sixth Embodiment

Now, the sixth embodiment for the present invention is described below.

Unlike the third embodiment, where the light irradiation means 74 in the DNA reading apparatus 70 irradiates the phosphor exciting light throughout on the surface of the optical DNA sensor 1 being attached to the apparatus 70, in the sixth embodiment, the light irradiation means in the DNA reading apparatus irradiates evanescent light as the phosphor exciting light from the close position throughout on the surface of the optical DNA sensor 1 attached to the apparatus 70.

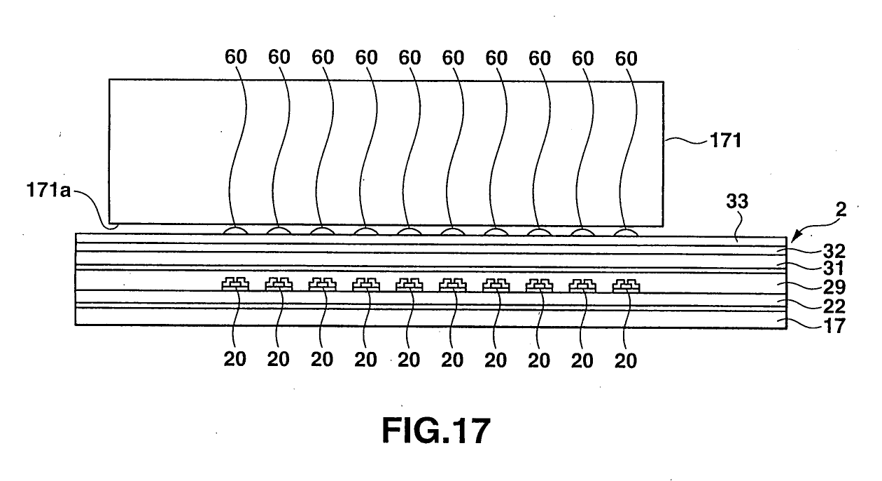

FIG. 17 is a side view showing the DNA reading apparatus according to the sixth embodiment. The light irradiation means of the DNA reading apparatus includes a light source (not shown) for emitting ultraviolet rays and a waveguide path 171 for transmitting ultraviolet rays emitted from the light source. Ultraviolet rays emitted from the light source are transmitted through the waveguide path 171 and are incident to a total reflection surface 171a of the waveguide path 171 at a critical angle or greater, and they are totally reflected there. In response to the total reflection, the evanescent light is projected from the total reflection surface 171a toward the outside of the waveguide path 171.

In the sixth embodiment as well, the optical DNA sensor 1 can be attached to or removed from the DNA reading apparatus. The surface of the optical DNA sensor 1 attached to the DNA reading apparatus faces to the total reflection surface 171a of the waveguide path 171, and the spots 60, 60, . . . are adjacent to the reverse surface 171a of the waveguide path 171.

Similarly to the DNA reading apparatus 70 according to the third embodiment, the DNA reading apparatus according to the sixth embodiment includes a display 3, an operation processor 4, a top gate driver 11, a bottom gate driver 12, a data driver 13 and a driving circuit 10.

In the DNA identification method using the DNA reading apparatus according to the sixth embodiment, similarly to the procedures described in the third embodiment, sample DNA segments labeled with fluorescent substance are hybridized with spots 60, 60, . . . of the optical DNA sensor 1, then followed by setting of the optical DNA sensor 1 to the DNA reading apparatus. Where, the spots 60, 60, . . . adjoin the total reflection surface 171a of the waveguide path 171, and following to turning on of the light source, evanescent light as the phosphor exciting light is irradiated from the total reflection surface 171a to the spots 60, 60, . . . Among the spots 60, 60, . . . , the ones having bonded with the sample DNA segments emit fluorescence, but the ones being not bonded with the sample DNA segments do not emit fluorescence. Then, the DNA reading apparatus causes the drivers 11, 12 and 13 and the driving circuit 10 to drive the optical DNA sensor 1 to acquire the fluorescence intensity distribution on the optical DNA sensor 1 as images of two dimensions. Following thereto, the images expressing the fluorescence intensity distribution are displayed by the operation processor 4 on the display 3. Depending on the portions in the displayed images where the light intensity is strong, nucleotide sequences in the sample DNA segments are determined.

As described above, in the sixth embodiment, since the evanescent light is hardly transmitted in a medium, it does not reach the semiconductor layers 23 of the sensors 20, 20, . . . As a result, only the semiconductor layers 23 of the sensors 20 that correspond to the spots 60 having bonded with the sample DNA segments are exposed. Hence, contrast of images expressing fluorescence intensity distribution is improved, and determination of nucleotide sequences in the sample DNA segments can be facilitated.

Note that the optical DNA sensors according to the fourth and fifth embodiments can be used for the DNA reading apparatus according to the sixth embodiment. In any case, it is needless to form the excited light absorbing layer 34, to reduce the charge density of the conductive layer 32 to a level of 1.0×10²⁰ [1/cm³] or less, and to laminate the dielectric multilayered film 35.

Besides, the light irradiation means for irradiating the evanescent light may be the one that includes a light source for emitting ultraviolet rays as parallel light and waveguide path plate that is plate-shaped and transmits the parallel light emitted from the light source in such a direction that the parallel light becomes parallel to a surface. In this case, when the optical DNA sensor 1 is attached to the DNA reading apparatus, the spots 60, 60, . . . are adjoined to the surface of the waveguide path. In response to the transmission of the parallel light through the waveguide path plate, the evanescent light is projected from the surface of the waveguide path plate to the outside and is incident to the spots 60, 60, . . .

### Seventh Embodiment

Now, the seventh embodiment for the present invention is described below.

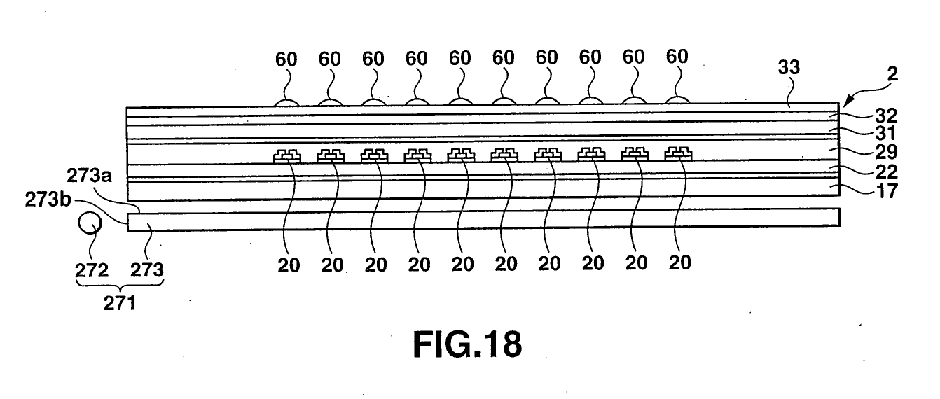

Unlike that layers for shading the phosphor exciting light, (that is, the excited light absorbing layer 34 and the dielectric multilayered film 35), are formed in an area extending from the semiconductor layer 23 to the spot 60 in the third and fifth embodiments, such layers for shading the phosphor exciting light are not formed in the optical DNA sensor according to the seventh embodiment, but the conductive layer 32 is formed directly on the protective insulated layer 31, as shown in FIG. 18. This conductive layer 32 has a charge density exceeding 1.0×10²⁰ [1/cm³] and is configured not to shade the phosphor exciting light, unlike the conductive layer described in the fourth embodiment. The other constituents of the optical DNA sensor according to the seventh embodiment are similar to those of the optical DNA sensor described in the third embodiment.

Furthermore, unlike the configuration of the third embodiment, where the light irradiation means 74 of the DNA reading apparatus 70 irradiates the phosphor exciting light against the surface of the solid imaging device 2, the light irradiation means 271 of the DNA reading apparatus irradiates the phosphor exciting light in a state like a plane of light throughout on the reverse surface of the attached solid imaging device 2 in the seventh embodiment.

The light irradiation means 271 of this DNA reading apparatus includes a light source 272 for emitting the phosphor exciting light and a light guide plate 273 for guiding the phosphor exciting light emitted from the light source 272 and irradiating it in a state like a plane of light from the surface 273a. The light guide plate 273 is substantially a flat plate-shaped and is coated with a reflective member except a side 272b facing to the light source 272 and the surface 273a.

In the seventh embodiment as well, the optical DNA sensor 1 is can be attached to and removed from the DNA reading apparatus, and it is configured such that the reverse surface of the solid imaging device 2 faces to the surface 273a of the light guide plate 273 when optical DNA sensor 1 is attached to the DNA reading apparatus. It is further configured such that, when the reverse surface of the solid imaging device 2 has faced to the surface 273a of the light guide plate 273, the phosphor exciting light like a plane of light projected from the surface 273a of the light guide plate 273 is uniformly irradiated to the reverse surface of the solid imaging device 2.

In such a configuration, the phosphor exciting light never be directly incident to the semiconductor layer 23 because the bottom gate electrodes 21 of the sensors 20, 20, . . . have shading property. Besides, the phosphor exciting light transmits through parts between the sensors 20, 20, . . . and is then incident to the spots 60, 60, . . . As a result, the spot 60 having bonded with the sample DNA segment emits fluorescence, and the fluorescence is incident to the semiconductor layer 23 of the sensor 20 corresponding to the spot 60.

As described above, in the seventh embodiment, the phosphor exciting light is shaded by the bottom gate electrodes 21 of the sensors 20, 20, . . . , while the fluorescence emitted from the spot 60 is not reflected and is incident to the semiconductor layer 23. As a result, only the semiconductor layer 23 of the sensor 20 corresponding to the spot 60 having bonded with the sample DNA segment is exposed. Therefore, contrast of images expressing fluorescence intensity distribution is improved, and determination of nucleotide sequences in the sample DNA segments can be facilitated.

Note that the present invention is not limited to the embodiments described above, and it will be appreciated that the present invention is susceptible to various modifications, and variations and changes in the design without departing from the proper scope and fair meaning of the present invention.

For example, although the spots 60, 60, . . . are directly fixed onto the overcoat layer 33, the overcoat layer 33 may not be formed on the conductive layer 32, and the spots 60, 60, . . . may be fixed directly onto the conductive layer 32. Further, the conductive layer 32 and the overcoat layer 33 may not be formed on the protective insulated layer 31, and the spots 60, 60, . . . may be fixed onto the protective insulated layer 31. Alternatively, instead of forming the conductive layer 32 on the protective insulated layer 31, the overcoat layer may be formed thereon, and the spots 60, 60, . . . may be fixed onto the overcoat layer 33.

In addition, in each of the above-described embodiments, although a positive voltage is impressed to the conductive layer 32, the voltage of the conductive layer may be set at a voltage of which absolute value is smaller than static electricity, for example 0 (V), so that the sensors 20, 20, . . . , and the top gate driver 11, the bottom gate driver 12, the data driver 13 and the driving circuit 10, those which are connected to a sensor 20, are protected from static electricity that is generated during a period throughout from the manufacturing of the solid imaging device 2 until the reading of DNA, to thereby cause the conductive layer to function as an electrode for discharging the static electricity.

Further, although the light irradiation means of the DNA reading apparatus 70 irradiates ultraviolet rays emitted in a state like a plane of light from the adjacent position as the excited light in each of the above-described embodiments, this excited light may be replaced with the evanescent light that is incident from a prefixed direction. In this case, since ultraviolet rays decline before reaching the semiconductor layer 23, even a semiconductor layer that is susceptible to excitation by ultraviolet rays may be used.

Further, the phosphor exciting light from the light source may not be totally reflected on the projection surface and may be directly incident from the projection surface to the surface of the optical DNA sensor 1. In this case, the surface of the optical DNA sensor needs not be adjacent to the projection surface.

Still further, the light irradiation means of the DNA reading apparatus 70 occasionally irradiates excited light against the surface of the optical DNA sensor in each of the above-described embodiments, the excited light may be irradiated from the reverse surface of the optical DNA sensor 1 against the reverse surface. In this case, since the bottom gate electrode 21 has shading property, the excited light never be directly incident to the semiconductor layer 23.

In each of the above-described embodiments, although the solid imaging device 2 using the sensors 20, 20, . . . as the photoelectric conversion element is exemplified for explanation of the present invention, the present invention may be applied to a solid imaging device using photodiodes as the photoelectric conversion elements. Examples of the solid imaging device using photodiodes include a CCD image sensor and a CMOS image sensor.

In the CCD image sensor, photodiodes are arrayed in a matrix fashion on a substrate. Around the photodiodes, a vertical CCS and a horizontal CCD, both for transmitting electric signals photoelectrically converted by the photodiodes are formed. In addition, like the foresaid solid imaging device 2, a protective insulated layer is formed throughout so as to coat a plurality of photodiodes, and a conductive layer is formed throughout on the protective insulated layer. Further, a plurality types of spots are arrayed on the conductive layer via the overcoat layer. When observing them in the plan view, one spot is superimposed on one photodiode, or some adjoining photodiodes constitute a set of photodiodes, and one spot is superimposed on the set of photodiodes.

In the CMOS image sensor, photodiodes are arrayed in a matrix fashion on a substrate. Around each of the photodiodes, a pixel circuit for amplifying electric signals photoelectrically converted by the photodiodes is provided. In addition, like the foresaid solid imaging device 2, a protective insulated layer is formed throughout so as to coat a plurality of photodiodes, and a conductive layer is formed throughout on the protective insulated layer. Further, a plurality types of spots are arrayed on the conductive layer via the overcoat layer. When observing them in the plan view, one spot is superimposed on one photodiode, or some adjoining photodiodes constitute a set of photodiodes, and one spot is superimposed on the set of photodiodes.

In each of the above-described embodiments, the sensor 20 of the double gate transistor type provided in the solid imaging device 2 is a transistor comprising a single channel amorphous silicon semiconductor layer. One pixel is constituted with only one sensor 20. Therefore, when using the photoelectric conversion element as a disposable sensor for the DNA identification, it is cheap to use this sensor comparing to the CCD image sensor and CMOS image sensor.

In each of the above-described embodiments, the mother substrate 35 was cut for each of the solid imaging device 2. However, a top gate driver 11, a bottom gate driver 12, a data driver 13 and a driving circuit 10, those which correspond to a plurality of solid imaging devices 2, may be provided to the DNA reading apparatus 70 to thereby perform DNA readings from the plurality of solid imaging devices 2 in the block.

Further, in each of the above-described embodiments, following to the attachment of the optical DNA sensor 1, to which a solution containing the sample DNA segment is applied, to the DNA reading apparatus 70, the top gate lines 44, 44, . . . are respectively connected to the terminals of the top gate driver 11, the bottom gate lines 41, 41, . . . are respectively connected to the terminals of the bottom gate driver 12, and the data lines 43, 43, . . . are respectively connected to the terminals of the data driver 13. However, before applying the solution containing the sample DNA segment to the optical DNA sensor 1, the top gate lines 44, 44, . . . , the bottom gate lines 41, 41, . . . and the data lines 43, 43, . . . may be connected in advance to the terminals of the top gate driver 11, the bottom gate driver 12 and the data driver 13, respectively.

Further, in each of the above-described embodiments, it is configured such that the sensor 20 is not sufficiently excited by ultraviolet rays but is sufficiently excited by visible light. However, the sensor 20 may be configured not to be sufficiently excited by visible light of a short wavelength but is sufficiently excited by visible light of a long wavelength. In conformity to such a configuration, it is allowable to select the fluorescent substance that absorbs visible light of a short wavelength and emits visible light with a long wavelength.

The spots 60, 60, . . . prepared in each of the above-described embodiments may be formed in the form of fine droplets by means of the ink-jet system to prefixed positions on the surface of the solid imaging device 2.

In the optical DNA sensors according to each of the above-described embodiments, although one sensor 20 corresponds to one spot 60, the spot 60 may be fixed on the surface of the solid imaging device 2 such that the spot 60 corresponds to the adjoining two or more sensors 20. However, it should be noted that any spot in the surface corresponds to the same number of sensors 20, and when “A” is the number of the sensors 20 included in one set (“A” is an integer of 2 or more) and “B” is the number of the sets, the number of the spots 60 comes to “B”, and the number of the sensors 20 included in the solid imaging device 2 is represented by (A×B).

1. An optical DNA sensor comprising:
a solid imaging device, and
a plurality types of DNA probe each including nucleotide sequence and being arrayed and fixed on a surface of the solid imaging device.

2. The optical DNA sensor as claimed in claim 1, wherein the solid imaging device comprises a plurality of photoelectric elements arranged on a substrate, and a transparent layer for coating the plurality of photoelectric elements, and the DNA probe are fixed on the transparent layer, corresponding to the photoelectric elements, respectively.

3. The optical DNA sensor as claimed in claim 1, wherein the solid imaging device comprises a plurality of photoelectric elements arranged on a substrate, and a transparent layer for coating the plurality of photoelectric elements, and each of the DNA probe is fixed on the transparent layer, corresponding to a group of adjacent photoelectric elements the number of which is “A” where “A” is an integer of 2 or more.

4. The optical DNA sensor as claimed in claim 2, wherein each of the photoelectric elements is of a field effect transistor type having a semiconductor layer which generates electric charges by receiving light.

5. An optical DNA sensor comprising:
a solid imaging device,
an excited light absorbing layer formed on a surface of the solid imaging device, and
a plurality types of DNA probe which include nucleotide sequence and are aligned and fixed on the excited light absorbing layer.

6. An optical DNA sensor comprising:
a solid imaging device,
a transparent conductive layer which is formed on a surface of the solid imaging device and has a charge density of 1.0×10²⁰ [1/cm3] or less, and
a plurality types of DNA probe which include nucleotide sequence and are aligned and fixed on the transparent conductive layer.

7. An optical DNA sensor comprising:
a solid imaging device;
a dielectric multilayered film comprising a plurality types of dielectric layers with refractive indexes different from each other, which are alternately laminated on a surface of the solid imaging device, an optical film thickness of each of the dielectric layers being equivalent to one fourth of a wavelength of a phosphor-exciting light; and
a plurality types of DNA probe which include nucleotide sequence and are aligned and fixed on the dielectric multilayered film.

8. An optical DNA sensor comprising:
a solid imaging device comprising: a plurality of photoelectric elements which are arranged apart from each other on a surface of a transparent substrate and include a bottom gate electrode 21 having a shading property, a semiconductor layer having a light sensitivity, a light-transmissive top gate electrode, which are layered on the transparent substrate in this order; and a light-transmissive protective layer for coating the plurality of photoelectric elements; and
a plurality types of DNA probe which include nucleotide sequence and are aligned and fixed on the protective layer.

9. A DNA reading apparatus comprising:
an optical DNA sensor comprising a solid imaging device, and a plurality types of DNA probe each including nucleotide sequence and being arrayed and fixed on a surface of the solid imaging device; and
a driving unit for attaching the optical DNA sensor detachably and for driving the solid imaging device.

10. A DNA reading apparatus comprising:
an optical DNA sensor which comprises:
a solid imaging device which comprises: a plurality of photoelectric elements which are arranged apart from each other on a surface of a transparent substrate and include a bottom gate electrode having a shading property, a semiconductor layer having a light sensitivity, a light-transmissive top gate electrode, which are layered on the transparent substrate in this order; and a light-transmissive protective layer for coating the plurality of photoelectric elements; and
a plurality types of DNA probe which include nucleotide sequence and are aligned and fixed on the protective layer; and
a light irradiation member for irradiating a phosphor exciting light like a plane of light toward a rear surface of the transparent substrate of the optical DNA sensor.

11. A DNA reading apparatus as claimed in claim 10, wherein the light irradiation member is disposed below the optical DNA sensor.

12. A DNA reading apparatus as claimed in claim 11, wherein the light irradiation member irradiates the phosphor exciting light to the DNA probe through the solid imaging device.

13. A DNA reading apparatus as claimed in claim 11, wherein the DNA probe is able to bond to an appropriate sample DNA having a fluorescent substance, the fluorescent substance is excited by the phosphor exciting light and emits a light is different in wavelength from the phosphor exciting light, the phosphor exciting light of the light irradiation member having a wavelength in a range which makes difficult for exciting the solid imaging device in comparison with the light emitted from the fluorescent substance.

14. A DNA identification method for identifying the sample DNA segment by using an optical DNA sensor, wherein the optical DNA sensor comprises:
a solid imaging device comprises a plurality of photoelectric elements arranged on a substrate, and a transparent layer for coating the plurality of photoelectric elements; and
a plurality types of DNA probe each including nucleotide sequence and being arrayed and fixed on a surface of the solid imaging device; and
the method comprising the steps of:
bonding a sample DNA segment to a complementary DNA probe among the plurality types of DNA probe by applying the sample DNA segment which was labeled with a fluorescent substance or a photoresonance scattering substance, on the transparent layer;
irradiating an exciting light to the plurality types of DNA probe; and
detecting an intensity of light from the fluorescent substance or the photoresonance scattering substance with the sample DNA segment bonded the complementary DNA probe.

15. A method for manufacturing a solid imaging device, comprising:
forming a conductive layer on a surface of a solid imaging device which comprises a plurality of photoelectric elements arranged on a substrate, and a transparent layer for coating the plurality of photoelectric elements; and
fixing DNA probe on a surface of the solid imaging device in a state of applying a voltage to the conductive layer.

16. The optical DNA sensor as claimed in claim 3, wherein each of the photoelectric elements is of a field effect transistor type having a semiconductor layer which generates electric charges by receiving light.

17. A DNA reading apparatus as claimed in claim 12, wherein the DNA probe is able to bond to an appropriate sample DNA having a fluorescent substance, the fluorescent substance is excited by the phosphor exciting light and emits a light is different in wavelength from the phosphor exciting light, the phosphor exciting light of the light irradiation member having a wavelength in a range which makes difficult for exciting the solid imaging device in comparison with the light emitted from the fluorescent substance.
